(19) United States
(12) Patent Application Publication
(10) Pub. No.: US 2025/0075203 A1
(43) Pub. Date: Mar. 6, 2025

(54) COMPOSITIONS AND METHODS FOR TARGETED MODIFICATION OF MSH3

(71) Applicant: Script Biosciences, Inc., Yardley, PA (US)

(72) Inventors: Ravi Ramakrishnan IYER, Wayne, PA (US); Ursula ANDRÉO, Ardmore, PA (US); Menggui HUANG, Drexel Hill, PA (US)

(21) Appl. No.: 18/821,690

(22) Filed: Aug. 30, 2024

Related U.S. Application Data
(60) Provisional application No. 63/580,322, filed on Sep. 1, 2023.

(51) Int. Cl. C12N 15/11; C07K 14/005; C12N 9/12; C12N 9/22; C12N 15/90

(52) U.S. Cl. CPC C12N 15/11; C07K 14/005; C12N 9/1252; C12N 9/22; C12N 15/907; C12Y 207/07007; C07K 2319/09; C12N 2310/20; C12N 2795/18022

(57) ABSTRACT

The subject disclosure provides CRISPR-based sequences, systems and compositions which can modify the exon region of certain disease-associated genes (e.g., MSH3) and decrease the expression thereof. The composition described herein can be used to treat patients who are at risk or show early symptoms of diseases associated with DNA-repeat expansion.

### CROSS-REFERENCE TO RELATED APPLICATIONS

This application claims the benefit of U.S. Provisional Application No. 63/580,322, filed Sep. 1, 2023, which is incorporated herein by reference in its entirety for all purposes.

### INCORPORATION BY REFERENCE OF SEQUENCE LISTING

The instant application contains a Sequence Listing which has been submitted electronically in XML file format and is hereby incorporated by reference in its entirety. Said XML copy, created on Aug. 29, 2024, is named 211507-013003_US_SL.xml and is 127,748 bytes in size.

### FIELD

The present disclosure relates to CRISPR-based gene editing technology applicable to treating diseases associated with DNA repeat expansion.

### BACKGROUND

Copy number changes within short tandem repeat sequences cause more than 30 human hereditary central nervous system (CNS) disorders. These include some of the more common neurodegenerative diseases, such as fragile X syndrome (FRAXA) and myotonic dystrophy type 1 (DM1), as well as rare ones such as Friedreich's ataxia (FRDA) and amyotrophic lateral sclerosis (ALS). The vast majority of such diseases are caused by expansions within (predominantly GC-rich) trinucleotide repeat tracts, although disease-causing instabilities of tetra-, penta-, hexa-, and dodecanucleotide repeats have been documented as well.

Although repeat expansion diseases are caused by a common type of mutation, the mechanisms underlying their pathogenesis vary significantly. On the basis of pathological mechanisms, triplet repeat disorders can be categorized as loss-of-function or gain-of-function diseases. FRAXA and FRDA are the best-characterized examples of loss-of-function diseases, wherein expansion of CGG⋅CCG and GAA⋅TTC repeats, respectively, results in epigenetic changes within the corresponding genes, culminating in heterochromatin-mediated transcriptional silencing of these genes. Thus, decreased levels of FMRP and frataxin polypeptides, respectively, are hallmarks of FRAXA and FRDA. By contrast, a large number of diseases are characterized by a toxic gain of function at the level of RNA (e.g., DM1, DM2, FXTAS, and FXPOI) and/or protein (e.g., HD and SBMA) (Iyer et al., Annu Rev Biochem.; 84:199-226 (2015)).

The significant impact and complex disease mechanism call for novel therapies for DNA expansion associated diseases.

### SUMMARY OF INVENTION

One aspect of the present disclosure provides a guide RNA comprising a first RNA sequence and a second RNA sequence, the first RNA sequence is capable of hybridizing to an exon of a human MSH3 gene, and the second RNA sequence comprises a Cas protein binding sequence.

In some embodiments, the exon is selected from exons 3, 4, 5, and 6.

In some embodiments, the first RNA sequence comprises a length of about 17 to about 23 nucleotides. In some specific embodiments, the first RNA sequence comprises a length of about 20 nucleotides.

In some embodiments, the first RNA sequence is the reverse complement to a target sequence in the exon.

In some embodiments, the first RNA sequence comprises a nucleotide sequence having at least 90%, 95%, 99%, or 100% sequence identity to any one of SEQ ID NOs: 1-6.

In some embodiments, the guide RNA comprises from 5′ to 3′: the first RNA sequence and the second RNA sequence.

In some embodiments, the second RNA further comprises one or more MS2 bacteriophage coat protein (MS2) binding sequence. In some further embodiments, the second RNA sequence comprises a nucleotide sequence having at least 90%, 95%, 99%, or 100% sequence identity to SEQ ID NO: 8 or 9.

In some embodiments, the guide RNA comprises a nucleotide sequence having at least 90%, 95%, 99%, or 100% sequence identity to any one of SEQ ID NOs: 10-23.

Another aspect of the disclosure provides a composition comprising: the guide RNA described herein, a Cas protein, or a recombinant nucleic acid encoding the Cas protein, and a T4 DNA polymerase segment, or a recombinant nucleic acid encoding the T4 DNA polymerase segment.

In some embodiments, the T4 DNA polymerase segment is a fusion protein comprising an MS2 bacteriophage coat protein.

In some embodiments, the fusion protein further comprises at least one nuclear localization signal sequence. In some further embodiments, the T4 DNA polymerase segment and the segment of the MS2 bacteriophage coat protein are separated by a first linker sequence.

In some embodiments, the composition further comprises a first linker amino acid sequence that links the MS2 bacteriophage coat protein to a first nuclear localization signal sequence, and a second linker sequence that links the T4 DNA polymerase segment to a second nuclear localization signal sequence.

In some embodiments, the Cas protein is Cas9 or a variant thereof.

In some embodiments, the recombinant nucleic acid encoding the Cas protein and/or the recombinant nucleic acid encoding the T4 DNA polymerase segment is an mRNA.

Another aspect of the present disclosure provides a recombinant nucleic acid comprising a first nucleic acid sequence that produces the guide RNA described herein, when transcribed.

In some embodiments, the recombinant nucleic acid further comprises a promoter operably linked to the first nucleic acid sequence. In some specific embodiments, the promoter is a U6 promoter.

In some embodiments, the recombinant nucleic acid further comprises a nucleotide “G” between the promoter and the first nucleic acid. In some specific embodiments, the recombinant nucleic acid produces higher level of guide RNA than a reference recombinant nucleic acid having the same sequence as the recombinant nucleic acid but without the nucleotide “G”.

In some embodiments, the recombinant nucleic acid further comprises a second nucleic acid sequence encoding a Cas protein and a fusion protein described herein.

In another aspect, the present disclosure provides an expression vector comprising the recombinant nucleic acid described herein. In some embodiments, the vector is a viral vector.

In another aspect, the present disclosure provides a cell comprising the recombinant nucleic acid described herein.

In yet another aspect, the present disclosure provides a pharmaceutical composition comprising the recombinant nucleic acid or the expression vector described herein, and a pharmaceutically acceptable carrier, diluent, or excipient.

In yet another aspect, the present disclosure provides a kit comprising the composition, the recombinant nucleic acid, or the expression vector described herein.

In another further aspect, the present disclosure provides a system for modifying a targeted genomic locus, said system comprising the guide RNA, the composition, the recombinant nucleic acid, or the expression vector described herein.

In yet another aspect, the present disclosure provides a method of treating a disease associated with DNA repeat expansion in a subject in need thereof, comprising introducing the composition of the recombinant nucleic acid, the expression vector, or the system described herein to the subject, such that the disease is treated.

In some embodiments, the introduction of the composition, the recombinant nucleic acid or the expression vector introduces a frameshift mutation in an exon of MSH3 in a cell of said subject.

In some embodiments, the introduction of the composition, the recombinant nucleic acid or the expression vector decreases the expression of MSH3 in a cell of said subject. In some embodiments, the expression level of MSH3 is decreased by at least 10%, 20%, 30%, 40%, 50%, 60%, 70%, 80%, 90%, 95%, 99%, or 100%. In some embodiments, the decrease in expression level of MSH3 in the cell of said subject comprises a decrease in the transcript level of MSH3 in the cell of said subject.

In some embodiments, the introduction of the composition, the recombinant nucleic acid or the expression vector does not change the expression of DHFR.

In some embodiments, the disease associated with DNA repeat expansion is any one of Huntington's disease (HD), myotonic dystrophy type 1 (DM1), fragile-X related disorders (FXDs), fragile XEMR (FRAXE), Friedrich's ataxia (FRDA), spinal and bulbar muscular atrophy (SBMA), spinocerebellar ataxia type 1 (SCA1), spinocerebellar ataxia type 8 (SCA8), and spinocerebellar ataxia type 12 (SCA12).

In some embodiments, the DNA repeat expansion is a trinucleotide repeat expansion.

### BRIEF DESCRIPTION OF DRAWINGS

FIGS. 1A-1C show the editing efficiency in HEK293 cells transfected with Cas9 (FIG. 1A), Cas-Plusv1 (FIG. 1B) or Cas-Plusv2 (FIG. 1C) for targeting exon 5 of the MSH3 gene using an sgRNA (Exon 5 SG 2; SEQ ID NO: 14). Results of 3 independent experiment are presented. The x axis is indel size spectrum, while the y axis is % of total edits, which indicate the editing frequency.

FIGS. 2A-2B show the editing efficiency in HEK293 cells transfected with Cas9, Cas-Plusv1 or Cas-Plusv2 for targeting exon 5 of the MSH3 gene using an sgRNA (Exon 5 SG 2; SEQ ID NO: 14; FIG. 2A) or exon 4 of the MSH3 gene using an sgRNA (Exon 4 SG 1; SEQ ID NO: 12; FIG. 2B). Results of 3 independent experiments are presented and compared for each set of guides (SEQ ID NOs: 12 and 14) are compared. The x axis is indel size spectrum, while the y axis is % of total edits, which indicate the editing frequency.

FIGS. 3A-3E show the editing efficiency in HEK293 cells transfected with Cas9 mRNA with or without T4 DNA polymerase v2 mRNA and sgRNA targeting exon 5 of the MSH3 gene (Exon 5 SG2; SEQ ID NO: 14). FIG. 3A shows the MSH3 editing efficiency as analyzed by TIDE analysis of Sanger sequencing data. The bar graph represents 3 independent experiments. The x axis represents the spectrum of indel sizes, while the y axis shows % of total edits, which indicates editing efficiency. FIG. 3B shows the MSH3 mRNA expression as quantified with qRT-PCR. The expression of MSH3 mRNA is normalized to GAPDH (n=7). FIG. 3C shows the MSH3 expression as analyzed by immunoblot assay of SDS-PAGE cell lysates of edited cells. The figure shows cell lysates harvested at different time points and blotted with antibodies directed at MS2-T4, Cas9, MSH3, DHFR, or GAPDH. FIG. 3D shows immunofluorescence microscopy images of MSH3 and MSH2 in HEK293 cells. FIG. 3E shows the results of PacBIO long read DNA sequencing of a 3782 bp PCR product spanning the editing site confirming the absence of large deletions at the MSH3 locus.

### DETAILED DESCRIPTION

The compositions, systems, and methods provided herein are based, at least in part, on the engineering and demonstration of efficiency of a novel CRISPR-based gene editing system including one or more components (e.g., guide RNA) described herein. The compositions, systems, and methods described herein can target a specific genomic locus and recruit a Cas protein, as well as other components, that facilitate the editing of the targeted genomic locus. Accordingly, the compositions, systems, and methods can be used to efficiently and specifically edit at least one genomic locus.

Certain mismatch repair genes (e.g., MSH2, MSH3, MLH1, PMS2, and MLH3) modify disease onset by promoting repeat expansion in a tissue-specific manner. Thus, by modulating (e.g., targeting and increasing and/or decreasing expression of) one or more targeting and decreasing the expression of a mismatch repair gene (e.g., MSH3) via introducing a composition, recombinant nucleic acid, or expression vector described herein, a disease associated with one or more mismatch repair genes (e.g., DNA repeat expansion-associated disease) can be treated. Accordingly, the compositions, systems, and methods described herein can be used to treat diseases associated with one or more mismatch repair genes, such as DNA-expansion diseases (e.g., myotonic dystrophy type 1, Huntington's disease etc.) by targeting one or more mismatch repair genes (e.g., MSH3).

### I. Definition

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. For example, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics can be combined in any suitable manner in one or more embodiments.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein by reference as they exist in the database on the filing date of this application or patent.

As used herein, the term “Cas protein” or “CRISPR-associated protein” refers to a nuclease which serves as the cleaving enzyme of a CRISPR-Cas system. Cas protein forms a ribonucleoprotein complex with a CRISPR RNA (crRNA) or a guide RNA, which surveys the genomic DNA and searches for sequences complementary to the crRNA spacer. The Cas protein cleaves the target DNA upon recognition.

Cas proteins are classified into different types and subtypes based on their structures, domain architectures, and functions. Non-limiting examples of Cas protein includes: Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4. Accordingly, CRISPR-Cas systems can be classified into different types. Three major types of Cas proteins are at the top of the classification hierarchy: type I CRISPR system enzyme (Cas3), type II CRISPR system enzyme (Cas9), and type III CRISPR system enzyme (Cas10). The signature gene for type II CRISPR system enzyme is Cas9, which encodes a multidomain protein that combines all the functions of effector complexes and the target DNA cleavage and is essential for the maturation of the crRNA. The large Cas9 protein (˜800-1,400 amino acids) contains two nuclease domains, namely the RuvC-like nuclease (RNase H fold) and the HNH (MerA-like) nuclease domain that is located in the middle of the protein. Both nucleases are required for target DNA cleavage (Makarova et al., Methods Mol Biol.; 1311:47-75 (2015)).

As used herein, the term “Cas protein binding sequence” refers to an RNA sequence that recruits or is bound by a Cas protein or Cas variant. A Cas protein binding sequence, including a Cas protein binding motif as described herein, can form part of a guide RNA or be part of a separate RNA molecule that interacts with the guide RNA. The Cas protein or variant thereof can bind to a Cas protein binding sequence resulting in a complex that recruits the Cas protein or variant thereof to a target DNA locus by the guide RNA.

As used herein, the term “Cas protein variant” refers to a mutant or a fragment of a wild type or naturally occurring Cas protein described herein that has enhanced, retained, reduced or lost activities, e.g., nuclease activity, as compared to the naturally occurring counterpart.

As used herein, the terms “guide RNA” and “gRNA” are used interchangeably and refer to an RNA polynucleotide that targets a Cas protein and/or other proteins (e.g., a fusion protein) to a specific DNA locus via base paring with the DNA locus. A guide RNA includes a nucleotide sequence, which can be referenced to herein as a “hybridization sequence,” that is complementary to or is the reverse complement of a target sequence in a DNA locus. Such a hybridization sequence allows the guide RNA to hybridized to the target sequence. A guide RNA can also include a Cas protein binding sequence at either end of the hybridization sequence (e.g., the 5′ end of the hybridization sequence). Such a guide RNA can be referred to as a single guide RNA (sgRNA). A guide RNA can also include other nucleotide sequences that perform different functions, such as recruiting other proteins (e.g., MS2 bacteriophage coat protein), or sequences that provide desired spacing between other components, such as a linker. A guide RNA can range in size depending upon the target sequence, the Cas protein system used for targeting the DNA locus, including the Cas protein binding sequence, and the other sequences present in the guide RNA. The secondary structure of a full RNA guide, including a Cas protein binding sequence, can be predicted using mfold (http://www.mfold.org/mfold/applications/rna-folding-form.php).

As used herein, the term “decrease the expression” or “decreasing the expression” refers to reducing the level of RNA or protein that is generated by a coding sequence, such as a gene, as compared to a reference level. The expression of a gene can be decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or completely knocked out. On the other hand, the term “increase the expression” or “increasing the expression” refers to increasing the level of RNA or protein that is generated by a coding sequence, such as a gene, as compared to a reference level. The expression of a gene can be increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 200%, at least 300%, at least 400%, at least 500%, or at least 1000%, or more.

As used herein, the terms “DNA repeat expansion,” “expanded nucleotide repeat,” “repeat expansion,” and “expanded polynucleotide repeat” are used interchangeably and refer to a mutation in which a normally polymorphic nucleotide repeat in a wild-type gene undergoes a mutational change whereby the repeat has expanded in length by the insertion of simple nucleotide repeats. This dynamic mutation is unlike conventional mutations because the expanded repeat can undergo further change, usually continued expansion, with each subsequent generation.

As used herein, the term “excipient” or “diluent” refers to an inactive substance formulated alongside the active ingredient of a medication, included for the purpose of long-term stabilization, bulking up solid formulations that contain potent active ingredients in small amounts or to confer a therapeutic enhancement on the active ingredient in the final dosage form, such as facilitating drug absorption, reducing viscosity, or enhancing solubility. Non-limiting examples of excipient include adjuvants, preservatives and vehicles.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides can be collectively referred to as gene product. If the polynucleotide is derived from genomic DNA, expression can include splicing of the mRNA in a eukaryotic cell. Accordingly, as used herein, the term “expression level” refers to the quantity or amount of product being generated by a polynucleotide that codes for an RNA or protein when transcribed and/or translated. Namely, the quantity or amount of the transcribed mRNA or other RNA transcript, and/or the translated peptides, polypeptides, or proteins of a polynucleotide.

As used herein, the term “exon” refers to any part of a gene that forms a part of the final mature RNA produced by that gene after introns have been removed by RNA splicing. In RNA splicing, introns are removed and exons are covalently joined to one another as part of generating the mature RNA.

As used herein, the term “frameshift mutation” refers to genetic mutation caused by an insertion or deletion of nucleotide bases in numbers that are not multiples of three. A frameshift mutation in an exon of a gene often leads to disruption of the codon reading frame, and either longer or shorter protein product of the gene than the normal protein.

As used herein, the term “fusion protein” refers to a polypeptide or protein comprising two or more separate proteins or polypeptides, which have the function of at least one of the original proteins, polypeptides or the fragments thereof. A linker (or spacer) peptide can be added between two neighboring polypeptides or proteins of different sources in the fusion protein.

As used herein, the term “hybridize,” “hybridization,” or “hybridizing” refers to a reaction or process in which one or more polynucleotides react to form a complex that is stabilized via hydrogen bonding between the bases of the nucleotide residues. The hydrogen bonding can occur by Watson Crick base pairing, Hoogsteen binding, or in any other sequence specific manner. Watson-Crick base-pairing includes: adenine (A) pairing with thymidine (T), adenine (A) pairing with uracil (U), and guanine (G) pairing with cytosine (C) for both DNA and RNA. For hybridization of a DNA molecule with an RNA molecule (e.g., when a DNA target nucleic acid base pairs with a guide RNA, etc.), guanine (G) can also base pair with uracil (U). Thus, a guanine (G) can be considered complementary to both an uracil (U) and to an adenine (A). The complex can comprise two strands forming a duplex structure, three or more strands forming a multi stranded complex, a single self-hybridizing strand, or any combination thereof. A hybridization reaction can constitute a step in a more extensive process, such as the initiation of PCR, or the cleavage of a polynucleotide by an enzyme. A sequence capable of hybridizing with a given target sequence can be referred to being “complementary” to the target sequence or as being the “reverse complement” of the given target sequence. Hybridization between two nucleotide sequences can depend on a number of conditions, including the length of the sequence and the degree of complementarity, variables that are well known in the art. Hybridization does not also require complete complementarity; there may be mismatches between the two nucleotide sequences. Any suitable in vitro assay can be utilized to assess whether two nucleotide sequences hybridize. One such assay is a melting point analysis, which assess the degree of complementarity between two nucleotide sequences by looking at the temperature to which the two nucleotide sequences disassociate. The greater the value of the melting temperature (Tm), the stronger the hybridization between the two nucleotide sequences. Typically, conditions of temperature and ionic strength are said to determine the “stringency” of the hybridization. Temperature, wash solution salt concentration, and other conditions can be adjusted as necessary according to factors such as length of the region of complementation and the degree of complementation, which are exemplified in Sambrook, J. and Russell, W., Molecular Cloning: A Laboratory Manual, Third Edition, Cold Spring Harbor Laboratory Press, Cold Spring Harbor (2001); and in Green, M. and Sambrook, J., Molecular Cloning: A Laboratory Manual, Fourth Edition, Cold Spring Harbor Laboratory Press, Cold Spring Harbor (2012).

As used herein, the term “linker” or “linker sequence” refers to a generally short peptide connecting neighboring polypeptides or proteins of a fusion protein. Linker is about 1 to about 50 amino acids in length. Empirical linkers designed by researchers are generally classified into 3 categories according to their structures: flexible linkers, rigid linkers, and in vivo cleavable linkers. Besides the basic role in linking the functional domains together (as in flexible and rigid linkers) or releasing free functional domain in vivo (as in in vivo cleavable linkers), linkers may offer many other advantages for the production of fusion proteins, such as improving biological activity, increasing expression yield, and achieving desirable pharmacokinetic profiles (Chen et al., Adv Drug Deliv Rev., 65 (10): 1357-1369 (2013)).

As used herein, the term “modulation” or “modulating,” in the context of a gene expression or its gene product, refers to changing (increasing or decreasing) the expression of a gene and/or the activity of a gene product. Modulating a gene can occur at various levels, including at genomic level, gene transcription, protein translation, post-translational modification, protein activity, etc. Modulating a gene can include complete knock-out of a gene, partially knock-out or knock-down of a gene, or completely or partially decrease the activity of the product encoded by the gene. In another instance, modulating a gene can substantially increase the expression of the gene or the activity of the product encoded by the gene. In another instance, modulating a gene can substantially decrease the expression of the gene or the activity of the product encoded by the gene.

As used herein, the term “MS2 binding sequence” or “MS2 bacteriophage coat protein binding sequence” refers to a nucleotide sequence that the MS2 bacteriophage coat protein is capable of specifically binding. Such a nucleotide sequence can be an RNA sequence that is contained within a guide RNA. A guide RNA can include one or more MS2 binding sequences, also referred to herein as MS2 binding motifs, as described herein. Any suitable MS2 binding sequence can be used that provides binding sites to the MS2 bacteriophage coat protein (see, e.g., Johansson et al., Seminars in Virology, 8 (3): 176-185 (1997)).

As used herein, the term “nuclear localization signal,” “nuclear localization sequence” or “NLS,” refers to short peptides that act as a signal fragment that mediates the transport of proteins from the cytoplasm into the nucleus. Nuclear localization signal is generally about 4 to about 40 amino acids in length. Nuclear localization signal can be classified into classical nuclear localization signal (cNLS), non-classical nuclear localization signal (ncNLS) and other types. Non-limiting examples of cNLS include PKLKRQ (SEQ ID NO: 63), RRARRPRG (SEQ ID NO: 64), GKRKLITSEEERSPAKRGRKS (SEQ ID NO: 65). Non-limiting examples of ncNLS include RSGGNHRRNGRGGRGGYNRRNNGYHPY (SEQ ID NO: 66), TLLLRETMNNLGVSDHAVLSRKTPQPY (SEQ ID NO: 67), and GKKKKGKPGKRREQRKKKRRT (SEQ ID NO: 68). Non-limiting examples of other types of NLS include RKHKTNRKPR (SEQ ID NO: 69), NRRAKAKR (SEQ ID NO: 70), RNKKKK (SEQ ID NO: 71) and RKVIK (SEQ ID NO: 72) (Lu et al., Cell Communication and Signaling; 19, 60 (2021)). Other examples of NLH sequences are described herein.

As used herein, the term “MS2 bacteriophage coat protein” refers to a polypeptide having the following sequence (also known as MS2 N55K variant) or an alternative MS2 bacteriophage coat protein:

(SEQ ID NO: 73)
MASNFTQFVLVDNGGTGDVTVAPSNFANGVAEWISSNSRSQAYKVTCSV
RQSSAQKRKYTIKVEVPKVATQTVGGVELPVAAWRSYLNMELTIPIFAT
NSDCELIVKAMQGLLKDGNPIPSAIAANSGIY

Alternative MS2 bacteriophage coat proteins that can be used herein includes at least 40%, at least 45%, at least 50%, at least 55%, at least 60%, at least 65%, at least 70%, at least 75%, at least 80%, at least 85%, at least 90%, at least 95%, at least 99%, or 100% sequence identity to the amino acid sequence set forth in SEQ ID NO: 73, and provides requisite binding to MS2 binding sequences.

As used herein, the term “on-site large indel” or “on-site large structural variants” refers to at least 50 bp insertions and deletions at the targeted site caused by, e.g., a targeted genomic editing technology such as CRISPR/Cas system. On-site large structural variants can pass to the next generation, and it's deemed undesirable in the art. (Hoijer et al., Nature commutations, 13, 627 (2022)).

As used herein, the terms “operably-linked,” “operatively linked,” “operably connected,” and “operatively connected” are used interchangeably and refer to the association of nucleic acid sequences on a single nucleic acid molecule (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory elements in a variety of orientations, including sense and antisense orientation.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. Polypeptides include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “percent sequence identity” or “sequence identity” refers to the degree of identity between any given query sequence and a subject sequence. A percent identity for any query nucleic acid or amino acid sequence, relative to another subject nucleic acid or amino acid sequence can be determined using tools and technologies known in the art, for example, NCBI BLAST tools, such as gapped BLAST, BLASTP, BLASTN, as well as ALIGN, FASTA and GCG, and other publicly and commercially available alignment algorithms and programs such as, but not limited to, ClustalW, Smith-Waterman in matlab, Bowtie, Gencious, Biopython and SeqMan. For the purposes of calculating percent sequence identity, thymine (T) may be considered the same as uracil (U).

As used herein, the term “pharmaceutically acceptable carrier” refers to any and all solvents, dispersion media, coatings, antibacterial and antifungal agents, isotonic and absorption delaying agents, buffers, and other excipients that are physiologically compatible. In some instances, the carrier is suitable for parenteral, oral, or topical administration, intramuscular injection, intravenous injection, or intracerebroventricular injection. Depending on the route of administration, the active compound, e.g., small molecule or biologic agent can be coated by a material to protect the compound from the action of acids and other natural conditions that can inactivate the compound.

As used herein, the terms “polynucleotide,” “nucleic acid,” and “nucleotide sequence” refer to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “recombinant nucleic acid” or “recombinant vector,” refers to polynucleotides or vector not found in the genome of a naturally occurring organism. Recombinant nucleic acids are often formed by genetic recombination (e.g., molecular cloning) that brings together genetic material from multiple sources. Recombinant vectors can include a nucleic acid described herein in a form suitable for expression of the nucleic acid in a host cell, which means that the recombinant vectors include one or more regulatory elements, which can be selected on the basis of the host cells to be used for expression, that is operatively-linked to the nucleic acid sequence to be expressed.

As used herein, the term “regulatory element,” refers to a promoter, enhancer, internal ribosomal entry sites (IRES), or other expression control element (e.g. transcription termination signals, such as polyadenylation signals and poly-U sequences).

As used herein, the term “scaffold RNA” refers to an RNA sequence that recruits or is bound by one or more proteins. A scaffold RNA can include a Cas protein binding sequence and/or other nucleotide sequences that are bound by other proteins, such as an MS2 binding sequence. A scaffold RNA can form part of a guide RNA or be part of a separate RNA molecule that interacts with the guide RNA. A Cas protein or variant thereof can bind to a Cas protein binding sequence within a scaffold RNA resulting in a complex that recruits the Cas protein or variant thereof to a target DNA locus by the guide RNA. A fusion protein described herein (e.g., a fusion protein containing a T4 DNA polymerase or a segment thereof, and optionally further containing an MS2 bacteriophage coat protein or a segment thereof) can bind to a scaffold RNA resulting in a complex that recruits the fusion protein to a target DNA locus by the guide RNA. In some instances, a scaffold RNA can include a combination of one or more Cas protein bind sequences and one or more RNA sequences that bind and recruit a fusion protein (e.g., MS2 binding sequences) that can result in a complex that recruits the Cas protein or variant thereof and a fusion protein described herein (e.g., a fusion protein containing an MS2 bacteriophage coat protein and a T4 DNA polymerase segment) to a target DNA locus by the guide RNA.

As used herein, the terms “subject” and “patient” are used interchangeably and refer to either a human or non-human, such as primates, mammals, and vertebrates. As used herein, a “subject in need thereof” or a “patient in need thereof,” refers to a subject or patient who is determined by methods and technologies known in the art that treatment or prevention of a disease, e.g., a disease associated with DNA repeat expansion, is needed. In some instances, patients include, but are not limited to human adults.

As used herein, the term “T4 DNA polymerase segment” refers to a polypeptide containing part of or the full sequence of T4 DNA polymerase, which has the following sequence:

(SEQ ID NO: 74)
KEFYISIETVGNNIVERYIDENGKERTREVEYLPTMFRHCKEESKYKDI
YGKNCAPQKFPSMKDARDWMKRMEDIGLEALGMNDFKLAYISDTYGSEI
VYDRKFVRVANCDIEVTGDKFPDPMKAEYEIDAITHYDSIDDRFYVFDL
LNSMYGSVSKWDAKLAAKLDCEGGDEVPQEILDRVIYMPFDNERDMLME
YINLWEQKRPAIFTGWNIEGFDVPYIMNRVKMILGERSMKRFSPIGRVK
SKLIQNMYGSKEIYSIDGVSILDYLDLYKKFAFTNLPSFSLESVAQHET
KKGKLPYDGPINKLRETNHQRYISYNIIDVESVQAIDKIRGFIDLVLSM
SYYAKMPFSGVMSPIKTWDAIIFNSLKGEHKVIPQQGSHVKQSFPGAFV
FEPKPIARRYIMSFDLTSLYPSIIRQVNISPETIRGQFKVHPIHEYIAG
TAPKPSDEYSCSPNGWMYDKHQEGIIPKEIAKVFFQRKDWKKKMFAEEM
NAEAIKKIIMKGAGSCSTKPEVERYVKFSDDFLNELSNYTESVLNSLIE
ECEKAATLANTNQLNRKILINSLYGALGNIHFRYYDLRNATAITIFGQV
GIQWIARKINEYLNKVCGTNDEDFIAAGDTDSVYVCVDKVIEKVGLDRF
KEQNDLVEFMNQFGKKKMEPMIDVAYRELCDYMNNREHLMHMDREAISC
PPLGSKGVGGFWKAKKRYALNVYDMEDKRFAEPHLKIMGMETQQSSTPK
AVQEALEESIRRILQEGEESVQEYYKNFEKEYRQLDYKVIAEVKTANDI
AKYDDKGWPGFKCPFHIRGVLTYRRAVSGLGVAPILDGNKVMVLPLREG
NPFGDKCIAWPSGTELPKEIRSDVLSWIDHSTLFQKSFVKPLAGMCESA
GMDYEEKASLDFLFG

A T4 DNA polymerase segment can include a variant T4 DNA polymerase have at least 40%, at least 45%, at least 50%, at least 55%, at least 60%, at least 65%, at least 70%, at least 75%, at least 80%, at least 85%, at least 90%, at least 95%, at least 99%, or 100% sequence identity to the amino acid sequence set forth in SEQ ID NO: 74, which retains the requisite T4 polymerase activity, and which can facilitate NHEJ. An example of a variant T4 DNA polymerase segment that is useful for the embodiments disclosed herein is a variant having the alteration D219A (located at position 218 in reference to SEQ ID NO: 74). In some embodiments, the variant T4 DNA polymerase segment has the amino acid sequence set forth in SEQ ID NO: 78.

The term “target sequence” as used herein, in the context of a DNA loci, refers to a nucleotide sequence found within the DNA loci. Such a nucleotide sequence can, for example, hybridize to a respective length portion of a hybridization sequence in a guide nucleic acid.

As used herein, the term “treating” or “treatment of” a disease, refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions can be administered to a subject at risk of developing a particular disease, condition, or symptom, or to a subject reporting one or more of the physiological symptoms of a disease, even though the disease, condition, or symptom may not have yet been manifested. Those in need of treatment include those already with disease. Hence, the patient to be treated herein may have been diagnosed as suffering from a disease, such as a disease associated with DNA repeat expansion. A disease is “inhibited” or “treated” if at least one symptom (as determined by responsiveness/non-responsiveness, or indicators known in the art and described herein) of the condition is alleviated, terminated, slowed, minimized, or prevented.

As used herein, the term “trinucleotide repeat expansion” refers to a series of three bases (e.g., CAG) repeated at least twice. For example, trinucleotide repeat expansions can be located in exons or introns of a gene. Trinucleotide repeat expansion is associated with a number of diseases such as myotonic dystrophy type 1 (DM1), Huntington's disease (HD), fragile-X related disorders (FXDs), fragile XE MR (FRAXE), Freidreich ataxia (FRDA), spinal and bulbar muscular atrophy (SBMA), spinocerebellar ataxia type 1 (SCA1), spinocerebellar ataxia type 8 (SCA8), and spinocerebellar ataxia type 12 (SCA12).

As used herein, the term “vector” or “expression vector,” refers to any nucleic acid construct capable of directing the delivery or transfer of a foreign genetic material to target cells, where it can be replicated and/or expressed. Thus, the term vector includes the construct to be delivered. Such a construct includes a macromolecule or complex of molecules having a polynucleotide to be delivered to a host cell, either in vitro or in vivo. A vector can be a linear or a circular molecule. A vector can be integrating or non-integrating. Vectors include, but are not limited to, nucleic acid molecules that are single-stranded, double-stranded, or partially double-stranded; nucleic acid molecules that have one or more free ends, no free ends (e.g., circular); nucleic acid molecules that have DNA, RNA, or both; and other varieties of polynucleotides known in the art. The major types of vectors include, but are not limited to, plasmids, episomal vectors, viral vectors, cosmids, and artificial chromosomes. One particular major type of vector is plasmid, which is a circular double stranded DNA loop into which additional DNA segments can be inserted, such as by standard molecular cloning techniques. Another major type of vector is viral vector, which includes virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

A vector can be designed based on factors such as the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

### II. CRISPR-Based Compositions and Systems

One aspect of the present disclosure provides a composition that includes components for CRISPR-based modification of one or more genomic locus. One aspect of the present disclosure provides a CRISPR-based system that is capable of modifying one or more genomic locus. The modification of the genomic loci can decrease the expression of the gene encoded by the genomic loci. In some embodiments, the composition can include a guide RNA described herein. In some embodiments, the composition can include a guide RNA described herein and one or more of the other components of the CRISPR-based system described herein. The system can include various components described herein, individually or in combination in a single composition. For example, the system can include a guide RNA described herein. In some embodiments, the system can further include a Cas protein as described herein. In some other embodiments, the system can further include a fusion protein as described herein. The genomic loci can encode one or more disease-associated gene (e.g., DNA expansion-associated genes). As such, targeting genomic loci encoding the disease-associated genes can be used to treat the corresponding DNA expansion-associated diseases.

### Guide RNA

A Guide RNA can include a hybridizing RNA sequence. The hybridizing RNA hybridizes to a target sequence in the disease-associated gene by reverse complement to the target sequence. Full complementarity is not necessarily required, provided there is sufficient complementarity to cause hybridization and promote formation of a CRISPR complex at a target loci. In some embodiments, the hybridizing RNA sequence is at least 70%, at least 75%, at least 80%, at least 85%, at least 90%, at least 95%, at least 99%, or 100% reverse complementary to the target sequence, and has sufficient complementarity to cause hybridization and promote formation of a CRISPR complex at the target loci. The ability of a hybridizing RNA sequence to direct sequence-specific binding of a CRISPR complex to a target sequence can be assessed by any suitable assay. For example, the components of a CRISPR system sufficient to form a CRISPR complex, including the hybridizing RNA sequence to be tested, can be provided to a host cell having the corresponding target sequence, such as by transfection with vectors encoding the components of the CRISPR complex, followed by an assessment of preferential cleavage within the target sequence, such as by Surveyor assay known in the art. Similarly, cleavage of a target polynucleotide sequence can be evaluated in a test tube by providing the target sequence, components of a CRISPR complex, including the hybridizing RNA sequence to be tested and a control hybridizing RNA sequence different from the test hybridizing RNA sequence, and comparing binding or rate of cleavage at the target sequence between the test and control hybridizing RNA sequence reactions.

In some embodiments, the hybridizing RNA sequence is also referred to as the first RNA sequence.

The hybridizing RNA sequence is selected to reduce the degree of secondary structure within the guide sequence. Secondary structure can be determined by any suitable polynucleotide folding algorithm. Some programs are based on calculating the minimal Gibbs free energy. An example of one such algorithm is mFold, as described by Zuker et al., Nucleic Acids Res., 9 (1): 133-148 (1981). Another example folding algorithm is the online webserver RNAfold, developed at Institute for Theoretical Chemistry at the University of Vienna, using the centroid structure prediction algorithm (see, e.g., Gruber et al., Cell 106 (1): 23-24 (2008); and Carr et al., Nature Biotechnology, 27 (12): 1151-62 (2009)). Further algorithms can be found in U.S. application Ser. No. 14/054,414; incorporated herein by reference.

Typically, in the context of an endogenous CRISPR system, formation of a CRISPR complex (including a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. In the present disclosure, the CRISPR-based system can include further components that facilitates genomic editing. In some embodiments, the system can include a guide RNA. In some further embodiments, the system includes a fusion protein described herein. The CRISPR-based system described herein can specifically and efficiently edit targeted genomic locus while decreasing on-site large indel or on-site large structural variants (Hoijer et al., Nature commutations, 13, 627 (2022)).

In some embodiments, the hybridizing RNA sequence has a length of about 15 to about 25 nucleotides. In some specific embodiments, the hybridizing RNA sequence has a length of about 15, about 16, about 17, about 18, about 19, about 20, about 21, about 22, about 23, about 24, or about 25 nucleotides. In some specific embodiments, the hybridizing RNA sequence has a length of about 20 nucleotides. In one embodiment, the hybridizing RNA sequence consists of a length of 20 nucleotides.

In one specific embodiment, the hybridizing RNA sequence is capable of hybridizing to an exon of a human disease-associated gene, e.g., at least one of exons 1-6 of human MSH3 gene. Accordingly, in some embodiments, the hybridizing RNA sequence capable of hybridizing to an exon of human MSH3 gene is reverse complement to a target sequence in one of exons 1-6 of human MSH3 gene.

Non-limiting examples of the hybridizing sequences are listed in Table 1, or the variants thereof. The variant sequences can have at least 80%, 85%, 90%, 95%, 99%, or 100% sequence identity to original sequence and encoding a product having the same function as the original sequence. In some embodiments, the hybridizing RNA sequence capable of hybridizing to an exon of human MSH3 gene has a nucleotide sequence having at least 90%, 95%, 99%, or 100% sequence identity to any one of SEQ ID NOs: 1-6. In some specific embodiments, the hybridizing RNA sequence capable of hybridizing to exon 3 of human MSH3 gene has a nucleotide sequence having at least 90%, 95%, 99%, or 100% sequence identity to SEQ ID NO: 1. In one specific embodiment, the hybridizing RNA sequence capable of hybridizing to exon 3 of human MSH3 gene has a nucleotide sequence identical to SEQ ID NO: 1. In some specific embodiments, the hybridizing RNA sequence capable of hybridizing to exon 4 of human MSH3 gene has a nucleotide sequence having at least 90%, 95%, 99%, or 100% sequence identity to SEQ ID NO: 2. In one specific embodiment, the hybridizing RNA sequence capable of hybridizing to exon 4 of human MSH3 gene has a nucleotide sequence identical to SEQ ID NO: 2. In some specific embodiments, the hybridizing RNA sequence capable of hybridizing to exon 5 of human MSH3 gene has a nucleotide sequence having at least 90%, 95%, 99%, or 100% sequence identity to any one of SEQ ID NOs: 3-5. In some specific embodiments, the hybridizing RNA sequence capable of hybridizing to exon 5 of human MSH3 gene has a nucleotide sequence identical to SEQ ID NOs: 3-5. In some specific embodiments, the hybridizing RNA sequence capable of hybridizing to exon 6 of human MSH3 gene has a nucleotide sequence having at least 90%, 95%, 99%, or 100% sequence identity to SEQ ID NO: 6. In one specific embodiment, the hybridizing RNA sequence capable of hybridizing to exon 6 of human MSH3 gene has a nucleotide sequence identical to SEQ ID NO: 6. In some embodiments, the hybridizing RNA sequence capable of hybridizing to exon 1 of human MSH3 gene. In some other embodiments, the hybridizing RNA sequence capable of hybridizing to exon 2 of human MSH3 gene.

The guide RNA can further include a scaffold sequence that recruits one or more components contributing to the modification of the targeted genomic locus. For example, the scaffold sequence can include a Cas protein binding sequence that recruit a Cas protein or a variant thereof to the targeted genomic loci. In some embodiments, a Cas protein binding sequence can be part of a separate RNA molecule that interacts with the guide RNA. The Cas protein or variant thereof can bind to a Cas protein binding sequence results in a complex that recruits the Cas protein or variant thereof to the target DNA locus.

The Cas protein binding sequence can be any RNA sequences known to bind at least one Cas protein or the variant thereof. Alternatively, the Cas protein binding sequence can be identified by methods known in the art. For example, a person skilled in the art can identify or screen for Cas protein binding sequences from an RNA library using assays such as biotinylated RNA pull-down analysis, RNA immunoprecipitation (RIP) analysis, RNA footprint analysis, and various UV crosslinking and immunoprecipitation. In some embodiments, the Cas protein binding sequence includes a nucleotide sequence having at least 90%, 95%, 99%, or 100% sequence identity to SEQ ID NO: 9. In one specific embodiment, the Cas protein binding sequence includes a nucleotide sequence identical to SEQ ID NO: 9.

The scaffold sequence can further include an RNA sequence that binds and recruits a fusion protein described herein to the target DNA locus.

The RNA sequence that binds and recruits a fusion protein can be any RNA sequences known to bind to the fusion protein. Alternatively, the RNA sequence can be identified by methods known in the art. For example, a person skilled in the art can identify or screen for the RNA sequences from an RNA library using assays such as biotinylated RNA pull-down analysis, RNA immunoprecipitation (RIP) analysis, RNA footprint analysis, and various UV crosslinking and immunoprecipitation.

In some embodiments, the RNA sequence that binds and recruits a fusion protein is an MS2 binding sequence. Accordingly, in some embodiments, the fusion protein includes a MS2 bacteriophage coat protein or a fragment or segment thereof. Any suitable MS2 bacteriophage coat protein binding sequence known in the art can be used herein. In addition, any MS2 bacteriophage coat protein binding sequence tested according to RNA-protein binding testing methods known in the art, e.g., those as described in Johansson et al., Seminars in Virology, 8 (3): 176-185 (1997), can be used herein. The scaffold RNA can further include 1, 2, 3, 4, 5, 6, 7, 8, 9, 10 or more of the MS2 bacteriophage coat protein binding sequences.

In some embodiments, the scaffold RNA sequence includes one or more Cas protein binding motifs, and one or more fusion protein binding motifs. In some specific embodiments, the scaffold RNA sequence includes a first Cas protein binding motif, a first MS2 binding motif, a second Cas protein binding motif, the first or a second MS2 binding motif, and a third Cas protein binding motif. In some specific embodiments, the scaffold RNA includes from 5′ to 3′ a first Cas protein binding motif, a first MS2 binding motif, a second Cas protein binding motif, the first or a second MS2 binding motif, and a third Cas protein binding motif.

Non-limiting examples of the scaffold RNA sequences are listed in Table 2, or the variants thereof. The variant sequences can have at least 80%, 85%, 90%, 95%, 99%, or 100% sequence identity to original sequence and encoding a product having the same function as the original sequence. In some specific embodiments, the scaffold RNA sequence has a nucleotide sequence having at least 90%, 95%, 99%, or 100% sequence identity to SEQ ID NO: 8. In one embodiment, the MS2 scaffold has a nucleotide sequence identical to SEQ ID NO: 8.

In some embodiments, the guide RNA includes a hybridizing RNA sequence described herein and a scaffold RNA sequence described herein. In some specific embodiments, the guide RNA includes from 5′ to 3′ a hybridizing RNA sequence and one or more scaffold RNA sequences. In some other embodiments, the guide RNA includes from 5′ to 3′ one or more scaffold RNA sequences and a hybridizing RNA sequence. In some other embodiments, the guide RNA includes a hybridizing RNA sequence, one or more scaffold RNA sequence, and additional RNA sequence in between.

In some embodiments, the scaffold RNA is also referred to the second RNA sequence.

Non-limiting examples of the guide RNA sequences are listed in Table 3, or the variants thereof. The variant sequences can have at least 80%, 85%, 90%, 95%, 99%, or 100% sequence identity to original sequence and encoding a product having the same function as the original sequence. In some embodiments, the guide RNA has a nucleotide sequence having at least 90%, 95%, 99%, or 100% sequence identity to any one of SEQ ID NOs: 10-23. In some embodiments, the guide RNA has a nucleotide sequence identical to any one of SEQ ID NOs: 10-23.

In some specific embodiments, the guide RNA includes a hybridizing RNA sequence having a nucleotide sequence having at least 90%, 95%, 99%, or 100% sequence identity to SEQ ID NO: 1, and a scaffold RNA sequence having a nucleotide sequence having at least 90%, 95%, 99%, or 100% sequence identity to SEQ ID NO: 8. In one specific embodiment, the guide RNA includes a hybridizing RNA sequence having a nucleotide sequence identical to SEQ ID NO: 1, and a scaffold RNA sequence having a nucleotide sequence identical to SEQ ID NO: 8. Accordingly, in some embodiments, the guide RNA has a nucleotide sequence identical to SEQ ID NO: 10. In some specific embodiments, the guide RNA includes a hybridizing RNA sequence having a nucleotide sequence having at least 90%, 95%, 99%, or 100% sequence identity to SEQ ID NO: 1, and a scaffold RNA sequence having a nucleotide sequence having at least 90%, 95%, 99%, or 100% sequence identity to SEQ ID NO: 9. In one specific embodiment, the guide RNA includes a hybridizing RNA sequence having a nucleotide sequence identical to SEQ ID NO: 1, and a scaffold RNA sequence having a nucleotide sequence identical to SEQ ID NO: 9. Accordingly, in some embodiments, the guide RNA has a nucleotide sequence identical to SEQ ID NO: 17.

In some specific embodiments, the guide RNA includes a hybridizing RNA sequence having a nucleotide sequence having at least 90%, 95%, 99%, or 100% sequence identity to SEQ ID NO: 2, and a scaffold RNA sequence having a nucleotide sequence having at least 90%, 95%, 99%, or 100% sequence identity to SEQ ID NO: 8. In one specific embodiment, the guide RNA includes a hybridizing RNA sequence having a nucleotide sequence identical to SEQ ID NO: 2, and a scaffold RNA sequence having a nucleotide sequence identical to SEQ ID NO: 8. Accordingly, in some embodiments, the guide RNA has a nucleotide sequence identical to SEQ ID NO: 11. In some specific embodiments, the guide RNA includes a hybridizing RNA sequence having a nucleotide sequence having at least 90%, 95%, 99%, or 100% sequence identity to SEQ ID NO: 2, and a scaffold RNA sequence having a nucleotide sequence having at least 90%, 95%, 99%, or 100% sequence identity to SEQ ID NO: 9. In one specific embodiment, the guide RNA includes a hybridizing RNA sequence having a nucleotide sequence identical to SEQ ID NO: 2, and a scaffold RNA sequence having a nucleotide sequence identical to SEQ ID NO: 9. Accordingly, in some embodiments, the guide RNA has a nucleotide sequence identical to SEQ ID NO: 18.

In some specific embodiments, the guide RNA includes a hybridizing RNA sequence having a nucleotide sequence having at least 90%, 95%, 99%, or 100% sequence identity to SEQ ID NO: 3, and a scaffold RNA sequence having a nucleotide sequence having at least 90%, 95%, 99%, or 100% sequence identity to SEQ ID NO: 8. In one specific embodiment, the guide RNA includes a second RNA sequence having a nucleotide sequence identical to SEQ ID NO: 3, and a scaffold RNA sequence having a nucleotide sequence identical to SEQ ID NO: 8. Accordingly, in some embodiments, the guide RNA has a nucleotide sequence identical to SEQ ID NO: 12. In some specific embodiments, the guide RNA includes a second RNA sequence having a nucleotide sequence having at least 90%, 95%, 99%, or 100% sequence identity to SEQ ID NO: 3, and a scaffold RNA sequence having a nucleotide sequence having at least 90%, 95%, 99%, or 100% sequence identity to SEQ ID NO: 9. In one specific embodiment, the guide RNA has a second RNA sequence having a nucleotide sequence identical to SEQ ID NO: 3, and a scaffold RNA sequence having a nucleotide sequence identical to SEQ ID NO: 9. Accordingly, in some embodiments, the guide RNA has a nucleotide sequence identical to SEQ ID NO: 19.

In some specific embodiments, the guide RNA includes a second RNA sequence having a nucleotide sequence having at least 90%, 95%, 99%, or 100% sequence identity to SEQ ID NO: 4, and a scaffold RNA sequence having a nucleotide sequence having at least 90%, 95%, 99%, or 100% sequence identity to SEQ ID NO: 8. In one specific embodiment, the guide RNA includes a second RNA sequence having a nucleotide sequence identical to SEQ ID NO: 4, and a scaffold RNA sequence having a nucleotide sequence identical to SEQ ID NO: 8. Accordingly, in some embodiments, the guide RNA has a nucleotide sequence identical to SEQ ID NO: 13. In some specific embodiments, the guide RNA includes a second RNA sequence having a nucleotide sequence having at least 90%, 95%, 99%, or 100% sequence identity to SEQ ID NO: 4, and a scaffold RNA sequence having a nucleotide sequence having at least 90%, 95%, 99%, or 100% sequence identity to SEQ ID NO: 9. In one specific embodiment, the guide RNA includes a second RNA sequence having a nucleotide sequence identical to SEQ ID NO: 4, and a scaffold RNA sequence having a nucleotide sequence identical to SEQ ID NO: 9. Accordingly, in some embodiments, the guide RNA has a nucleotide sequence identical to SEQ ID NO: 20.

In some specific embodiments, the guide RNA includes a second RNA sequence having a nucleotide sequence having at least 90%, 95%, 99%, or 100% sequence identity to SEQ ID NO: 5, and a scaffold RNA sequence having a nucleotide sequence having at least 90%, 95%, 99%, or 100% sequence identity to SEQ ID NO: 8. In one specific embodiment, the guide RNA includes a second RNA sequence having a nucleotide sequence identical to SEQ ID NO: 5, and a scaffold RNA sequence having a nucleotide sequence identical to SEQ ID NO: 8. Accordingly, in some embodiments, the guide RNA has a nucleotide sequence identical to SEQ ID NO: 14. In some specific embodiments, the guide RNA includes a second RNA sequence having a nucleotide sequence having at least 90%, 95%, 99%, or 100% sequence identity to SEQ ID NO: 5, and a scaffold RNA sequence having a nucleotide sequence having at least 90%, 95%, 99%, or 100% sequence identity to SEQ ID NO: 9. In one specific embodiment, the guide RNA includes a second RNA sequence having a nucleotide sequence identical to SEQ ID NO: 5, and a scaffold RNA sequence having a nucleotide sequence identical to SEQ ID NO: 9. Accordingly, in some embodiments, the guide RNA has a nucleotide sequence identical to SEQ ID NO: 21.

In some specific embodiments, the guide RNA includes a second RNA sequence having a nucleotide sequence having at least 90%, 95%, 99%, or 100% sequence identity to SEQ ID NO: 6, and a scaffold RNA sequence having a nucleotide sequence having at least 90%, 95%, 99%, or 100% sequence identity to SEQ ID NO: 8. In some specific embodiments, the guide RNA includes a second RNA sequence having a nucleotide sequence having at least 90%, 95%, 99%, or 100% sequence identity to SEQ ID NO: 6, and a scaffold RNA sequence having a nucleotide sequence having at least 90%, 95%, 99%, or 100% sequence identity to SEQ ID NO: 8. In some specific embodiments, the guide RNA include a second RNA sequence having a nucleotide sequence having at least 90%, 95%, 99%, or 100% sequence identity to SEQ ID NO: 15, and a scaffold RNA sequence having a nucleotide sequence having at least 90%, 95%, 99%, or 100% sequence identity to SEQ ID NO: 9. In one specific embodiment, the guide RNA includes a second RNA sequence having a nucleotide sequence identical to SEQ ID NO: 6, and a scaffold RNA sequence having a nucleotide sequence identical to SEQ ID NO: 9. Accordingly, in some embodiments, the guide RNA has a nucleotide sequence identical to SEQ ID NO: 22.

### Other Components of the CRISPR-Based System

In some aspects of the present disclosure, the CRISPR-based system includes a Cas protein or variants thereof. The Cas protein or variants thereof is recruited to the targeted genomic locus by the scaffold RNA sequence, and excise the genomic DNA at or near the locus.

Any Cas protein or variants thereof can be used in the composition. In some embodiments, the CRISPR enzyme directs cleavage of one or two strands at the location of the target sequence. Non-limiting examples of the Cas protein that can be useful in the compositions and methods described herein include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and the variants thereof. The sequences of these enzymes are known, for example, from public databases such as SwissProt. In some specific embodiments, the Cas protein is a type II CRISPR system enzyme. In some embodiments, the Cas protein is a Cas9 enzyme. In some embodiments, the Cas9 enzyme is S. pneumoniae, S. pyogenes, or S. thermophilus Cas9, and can include mutated Cas9 derived from these organisms. The Cas protein can be a Cas9 homolog or ortholog. The amino acid sequence of S. pyogenes Cas9 protein can be found in the SwissProt database under accession number Q99ZW2.

In some embodiments, the Cas protein has DNA cleavage activity, such as Cas9. In some embodiments, the Cas protein directs cleavage of one or both strands at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, the Cas protein is a variant with respect to a corresponding wild-type enzyme such that the Cas protein variant lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A.

In some embodiments, a nucleic acid having a coding sequence of a Cas protein described herein is codon optimized for expression in a particular cell, such as a eukaryotic cell. The eukaryotic cell can be any one of those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database,” and these tables can be adapted in a number of ways (see, e.g., Nakamura, et al., Nucl. Acids Res., 28 (1): 292 (2000)). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

In some embodiments, the CRISPR-based system further includes a first protein or polypeptide that reduces on-target DNA damage of CRISPR/Cas-mediated genome editing such as those described in Yang et al. (bioRxiv, doi.org/10.1101/2023.01.10.523496 (January 2023)). In some embodiments, the first protein or polypeptide is a DNA polymerase or a segment thereof. In some embodiments, the DNA polymerase or a segment thereof is a T4 DNA polymerase or a segment thereof. Non-limiting examples of the first protein or polypeptide include the T4 DNA polymerase and the segments thereof as disclosed in Yang et al. (bioRxiv, doi.org/10.1101/2023.01.10.523496 (January 2023)) and International Publication No. WO 2022/098923. In some embodiments, the first protein or polypeptide alone can be present at the target DNA locus. In some embodiments, the T4 DNA polymerase further includes a nuclear localization signal (NLS). In some embodiments, the T4 DNA polymerase is encoded by a nucleic acid sequence as set forth in SEQ ID NO: 77. In some embodiments, the T4 DNA polymerase has an amino acid sequence as set forth in SEQ ID NO: 78 combined with NLS sequences described herein, resulting in an amino acid sequence having at least 80%, 85%, 90%, 95%, 99%, or 100% sequence identity to SEQ ID NO: 79.

In some embodiments, the first protein or polypeptide is a fusion protein further including a second protein or polypeptide that can specifically bind a scaffold RNA sequence. As a result, in some embodiments, the fusion protein is recruited to the targeted locus. The second protein or polypeptide and the portion of the scaffold RNA interacting with it can be any known RNA-protein interaction pairs. For example, a person skilled in the art can test any RNA-binding proteins known in the art and adapt the suitable pairs to be used as the second protein or polypeptide, and the corresponding scaffold RNA sequence.

In some embodiments, the second protein or polypeptide is a bacteriophage coat protein or a segment thereof. In some specific embodiments, the second protein or polypeptide is an MS2 bacteriophage coat protein or a segment thereof, such as those disclosed in Yang et al. (bioRxiv, doi.org/10.1101/2023.01.10.523496 (January 2023)) and international publication No. WO 2022/098923, content of which are incorporated herein by reference.

In some embodiments, the fusion protein described herein further comprises a linker that links the first and second proteins or polypeptides. In some embodiments, the fusion protein further comprises one or more (e.g., 1, 2, 3, 4, or more) nuclear localization signal (NLS) sequences. In some embodiments, the fusion protein further comprises a first linker linking a first NLS sequence to a first protein or polypeptide, and a second linker linking a second NLS sequence to a second protein or polypeptide. An exemplary fusion protein is described in Table 7 and SEQ ID NO: 79.

Any known peptide linker in the art can be used herein. In some embodiments, the linker is 4-100 amino acids in length, for example, about 4, about 6, about 8, about 10, about 12, about 14, about 16, about 18, about 20, about 25, about 30, about 35, about 40, about 45, about 50, about 55, about 60, about 65, about 70, about 75, about 80, about 85, about 90, about 95, or about 100 amino acids in length. Longer or shorter linkers are also contemplated. In some specific embodiments, a linker comprises the amino acid sequence SGSETPGTSESATPES (SEQ ID NO: 52), which is also referred to as the XTEN linker, SGSETPGTSESA (SEQ ID NO: 53), SGSETPGTSESATPEGGSGGS (SEQ ID NO: 54), VPFLLEPDNINGKTC (SEQ ID NO: 55), GSAGSAAGSGEF (SEQ ID NO: 56), SIVAQLSRPDPA (SEQ ID NO: 57), MKIIEQLPSA (SEQ ID NO: 58), VRHKLKRVGS (SEQ ID NO: 59), GHGTGSTGSGSS (SEQ ID NO: 96), MSRPDPA (SEQ ID NO: 60), or GGSM (SEQ ID NO: 61). In some specific embodiments, a linker comprises the amino acid sequence SGGS (SEQ ID NO: 62). In some embodiments, a linker comprises (GGS) n (SEQ ID NO: 97), (SGGS) n (SEQ ID NO: 80), (GGGS) n (SEQ ID NO: 81), (GGGGS) n (SEQ ID NO: 82), (G) n (SEQ ID NO: 83), (EAAAK) n (SEQ ID NO: 84), (GGS) n (SEQ ID NO: 97) or (XP) n (SEQ ID NO: 85) motif, or a combination of any of these, where n is independently an integer between 1 and 30, and where X is any amino acid. In some embodiments, n is 1, 2, 3, 4, 5, 6, 7, 8, 9, 10, 11, 12, 13, 14, or 15 (International Publication No. WO2018031683, the content of which is incorporated herein by reference). In some embodiments, a linker comprises an amino acid sequence for the linkers found within SEQ ID NO: 79.

The NLS sequence can be any NLS known in the art. For example, a plurality of NLS sequences are described in international publication No. WO 2001/038547, and U.S. Pat. No. 9,278,067, the contents of which are incorporated herein by reference for their disclosure of exemplary NLS sequences. Other exemplary NLS sequences are found within SEQ ID NO: 79, any one of which can be used as a NLS sequence as described herein. In some embodiments, the fusion protein comprises a DNA polymerase or a segment thereof and an NLS as described herein. In some embodiments the DNA polymerase or the segment thereof is a T4 DNA polymerase or a segment thereof. In some embodiments, the T4 DNA polymerase is encoded by a nucleic acid sequence as set forth in SEQ ID NO: 77. In some embodiments, the T4 DNA polymerase has an amino acid sequence as set forth in SEQ ID NO: 78. In some embodiments, the fusion protein comprises an amino acid sequence having at least 80%, 85%, 90%, 95%, 99%, or 100% sequence identity to SEQ ID NO: 79.

Non-limiting examples of a fusion protein include Cas-Plusv1 and Cas-Plusv2 as described in International Publication No. WO 2022/098923.

### Nucleic Acid Encoding the CRISPR-Based System

Some further aspects of the present disclosure provide a recombinant nucleic acid producing or encoding one or more components of the CRISPR-based system described herein. For example, a recombinant nucleic acid can include a first nucleic acid sequence that produces a guide RNA described herein, when transcribed.

Non-limiting examples of the first nucleic acid sequences are those listed in Table 4, or the variants thereof. The variant sequences can have at least 80%, 85%, 90%, 95%, 99%, or 100% sequence identity to original sequence and encoding a product having the same function as the original sequence. In some embodiments, the first nucleic acid sequence has a nucleotide sequence having at least 80%, 85%, 90%, 95%, 99%, or 100% sequence identity to any one of SEQ ID NOs: 24-37. In some specific embodiments, the first nucleic acid sequence has a nucleotide sequence identical to any one of SEQ ID NOs: 24-37.

In some specific embodiments, the first nucleic acid sequence has a nucleotide sequence having at least 80%, 85%, 90%, 95%, 99%, or 100% sequence identity to SEQ ID NO: 24. In one specific embodiment, the first nucleic acid sequence has a nucleotide sequence identical to SEQ ID NO: 24. In some specific embodiments, the first nucleic acid sequence has a nucleotide sequence having at least 80%, 85%, 90%, 95%, 99%, or 100% sequence identity to SEQ ID NO: 25. In one specific embodiment, the first nucleic acid sequence has a nucleotide sequence identical to SEQ ID NO: 25. In some specific embodiments, the first nucleic acid sequence has a nucleotide sequence having at least 80%, 85%, 90%, 95%, 99%, or 100% sequence identity to SEQ ID NO: 26. In one specific embodiment, the first nucleic acid sequence has a nucleotide sequence identical to SEQ ID NO: 26. In some specific embodiments, the first nucleic acid sequence has a nucleotide sequence having at least 80%, 85%, 90%, 95%, 99%, or 100% sequence identity to SEQ ID NO: 27. In one specific embodiment, the first nucleic acid sequence has a nucleotide sequence identical to SEQ ID NO: 27. In some specific embodiments, the first nucleic acid sequence has a nucleotide sequence having at least 80%, 85%, 90%, 95%, 99%, or 100% sequence identity to SEQ ID NO: 28. In one specific embodiment, the first nucleic acid sequence has a nucleotide sequence identical to SEQ ID NO: 28. In some specific embodiments, the first nucleic acid sequence has a nucleotide sequence having at least 80%, 85%, 90%, 95%, 99%, or 100% sequence identity to SEQ ID NO: 29. In one specific embodiment, the first nucleic acid sequence has a nucleotide sequence identical to SEQ ID NO: 29. In some specific embodiments, the first nucleic acid sequence has a nucleotide sequence having at least 80%, 85%, 90%, 95%, 99%, or 100% sequence identity to SEQ ID NO: 30. In one specific embodiment, the first nucleic acid sequence has a nucleotide sequence identical to SEQ ID NO: 30. In some specific embodiments, the first nucleic acid sequence has a nucleotide sequence having at least 80%, 85%, 90%, 95%, 99%, or 100% sequence identity to SEQ ID NO: 31. In one specific embodiment, the first nucleic acid sequence has a nucleotide sequence identical to SEQ ID NO: 31. In some specific embodiments, the first nucleic acid sequence has a nucleotide sequence having at least 80%, 85%, 90%, 95%, 99%, or 100% sequence identity to SEQ ID NO: 32. In one specific embodiment, the first nucleic acid sequence has a nucleotide sequence identical to SEQ ID NO: 32. In some specific embodiments, the first nucleic acid sequence has a nucleotide sequence having at least 80%, 85%, 90%, 95%, 99%, or 100% sequence identity to SEQ ID NO: 33. In one specific embodiment, the first nucleic acid sequence has a nucleotide sequence identical to SEQ ID NO: 33. In some specific embodiments, the first nucleic acid sequence has a nucleotide sequence having at least 80%, 85%, 90%, 95%, 99%, or 100% sequence identity to SEQ ID NO: 34. In one specific embodiment, the first nucleic acid sequence has a nucleotide sequence identical to SEQ ID NO: 34. In some specific embodiments, the first nucleic acid sequence has a nucleotide sequence having at least 80%, 85%, 90%, 95%, 99%, or 100% sequence identity to SEQ ID NO: 35. In one specific embodiment, the first nucleic acid sequence has a nucleotide sequence identical to SEQ ID NO: 35. In some specific embodiments, the first nucleic acid sequence has a nucleotide sequence having at least 80%, 85%, 90%, 95%, 99%, or 100% sequence identity to SEQ ID NO: 36. In one specific embodiment, the first nucleic acid sequence has a nucleotide sequence identical to SEQ ID NO: 36. In some specific embodiments, the first nucleic acid sequence has a nucleotide sequence having at least 80%, 85%, 90%, 95%, 99%, or 100% sequence identity to SEQ ID NO: 37. In one specific embodiment, the first nucleic acid sequence has a nucleotide sequence identical to SEQ ID NO: 37.

In some embodiments, the recombinant nucleic acid further includes a regulatory element, e.g., a promoter, operably linked to the first nucleic acid sequence to facilitate the expression of the first nucleic acid in a host cell.

Regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements can also direct expression in a temporal-dependent manner, such as in a cell-cycle dependent or developmental stage-dependent manner, which may or may not also be tissue or cell-type specific. In some embodiments, a vector has one or more RNA polymerase III promoter, one or more RNA polymerase II promoter, or one or more RNA polymerase I promoter. Examples of RNA polymerase III promoters include, but are not limited to, U6 and H1 promoters. Examples of RNA polymerase II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the β-actin promoter, the phosphoglycerol kinase (PGK) promoter, and the EF1α promoter. In some embodiments, the regulatory element further includes one or more enhancer elements, such as WPRE; CMV enhancers; the R-U5′ segment in LTR of HTLV-I (Takebe et al., Mol. Cell. Biol., 8 (1), 466-472, (1988)); SV40 enhancer; and the intron sequence between exons 2 and 3 of rabbit β-globin (O'Hare et al., Proc. Natl. Acad. Sci. USA, 78 (3): 1527-31 (1981)).

In some specific embodiments, the regulatory element includes a U6 promoter, accordingly, the recombinant nucleic acid includes a first nucleic acid operably linked to a U6 promoter. In some specific embodiments, the recombinant nucleic acid further includes a nucleotide “G” between the U6 promoter and the first nucleic acid. The recombinant nucleic acid with the nucleotide “G” between the U6 promoter and the first nucleic acid produces higher level (e.g., at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000% or higher level) of guide RNA than a reference recombinant nucleic acid having the same sequence as the recombinant nucleic acid but without the nucleotide “G”.

Non-limiting examples of the recombinant nucleic acid sequences are those listed in Table 5, or the variants thereof. The variant sequences can have at least 80%, 85%, 90%, 95%, 99%, or 100% sequence identity to original sequence and encoding a product having the same function as the original sequence. In some embodiments, the recombinant nucleic acid has a nucleotide sequence having at least 80%, 85%, 90%, 95%, 99%, or 100% sequence identity to any one of SEQ ID NOs: 38-51. In some specific embodiments, the recombinant nucleic acid has a nucleotide sequence identical to any one of SEQ ID NOs: 38-51.

In some specific embodiments, the recombinant nucleic acid has a nucleotide sequence having at least 80%, 85%, 90%, 95%, 99%, or 100% sequence identity to SEQ ID NO: 38. In one specific embodiment, the recombinant nucleic acid has a nucleotide sequence having at least 80%, 85%, 90%, 95%, 99%, or 100% sequence identity to SEQ ID NO: 38. In some specific embodiments, the recombinant nucleic acid has a nucleotide sequence having at least 80%, 85%, 90%, 95%, 99%, or 100% sequence identity to SEQ ID NO: 39. In one specific embodiment, the recombinant nucleic acid has a nucleotide sequence having at least 80%, 85%, 90%, 95%, 99%, or 100% sequence identity to SEQ ID NO: 39. In some specific embodiments, the recombinant nucleic acid has a nucleotide sequence having at least 80%, 85%, 90%, 95%, 99%, or 100% sequence identity to SEQ ID NO: 40. In one specific embodiment, the recombinant nucleic acid has a nucleotide sequence having at least 80%, 85%, 90%, 95%, 99%, or 100% sequence identity to SEQ ID NO: 40. In some specific embodiments, the recombinant nucleic acid has a nucleotide sequence having at least 80%, 85%, 90%, 95%, 99%, or 100% sequence identity to SEQ ID NO: 41. In one specific embodiment, the recombinant nucleic acid has a nucleotide sequence having at least 80%, 85%, 90%, 95%, 99%, or 100% sequence identity to SEQ ID NO: 41. In some specific embodiments, the recombinant nucleic acid has a nucleotide sequence having at least 80%, 85%, 90%, 95%, 99%, or 100% sequence identity to SEQ ID NO: 42. In one specific embodiment, the recombinant nucleic acid has a nucleotide sequence having at least 80%, 85%, 90%, 95%, 99%, or 100% sequence identity to SEQ ID NO: 42. In some specific embodiments, the recombinant nucleic acid has a nucleotide sequence having at least 80%, 85%, 90%, 95%, 99%, or 100% sequence identity to SEQ ID NO: 43. In one specific embodiment, the recombinant nucleic acid has a nucleotide sequence having at least 80%, 85%, 90%, 95%, 99%, or 100% sequence identity to SEQ ID NO: 43. In some specific embodiments, the recombinant nucleic acid has a nucleotide sequence having at least 80%, 85%, 90%, 95%, 99%, or 100% sequence identity to SEQ ID NO: 44. In one specific embodiment, the recombinant nucleic acid has a nucleotide sequence having at least 80%, 85%, 90%, 95%, 99%, or 100% sequence identity to SEQ ID NO: 44. In some specific embodiments, the recombinant nucleic acid has a nucleotide sequence having at least 80%, 85%, 90%, 95%, 99%, or 100% sequence identity to SEQ ID NO: 45. In one specific embodiment, the recombinant nucleic acid has a nucleotide sequence having at least 80%, 85%, 90%, 95%, 99%, or 100% sequence identity to SEQ ID NO: 45. In some specific embodiments, the recombinant nucleic acid has a nucleotide sequence having at least 80%, 85%, 90%, 95%, 99%, or 100% sequence identity to SEQ ID NO: 46. In one specific embodiment, the recombinant nucleic acid has a nucleotide sequence having at least 80%, 85%, 90%, 95%, 99%, or 100% sequence identity to SEQ ID NO: 46. In some specific embodiments, the recombinant nucleic acid has a nucleotide sequence having at least 80%, 85%, 90%, 95%, 99%, or 100% sequence identity to SEQ ID NO: 47. In one specific embodiment, the recombinant nucleic acid has a nucleotide sequence having at least 80%, 85%, 90%, 95%, 99%, or 100% sequence identity to SEQ ID NO: 47. In some specific embodiments, the recombinant nucleic acid has a nucleotide sequence having at least 80%, 85%, 90%, 95%, 99%, or 100% sequence identity to SEQ ID NO: 48. In one specific embodiment, the recombinant nucleic acid has a nucleotide sequence having at least 80%, 85%, 90%, 95%, 99%, or 100% sequence identity to SEQ ID NO: 48. In some specific embodiments, the recombinant nucleic acid has a nucleotide sequence having at least 80%, 85%, 90%, 95%, 99%, or 100% sequence identity to SEQ ID NO: 49. In one specific embodiment, the recombinant nucleic acid has a nucleotide sequence having at least 80%, 85%, 90%, 95%, 99%, or 100% sequence identity to SEQ ID NO: 49. In some specific embodiments, the recombinant nucleic acid has a nucleotide sequence having at least 80%, 85%, 90%, 95%, 99%, or 100% sequence identity to SEQ ID NO: 40. In one specific embodiment, the recombinant nucleic acid has a nucleotide sequence having at least 80%, 85%, 90%, 95%, 99%, or 100% sequence identity to SEQ ID NO: 50. In some specific embodiments, the recombinant nucleic acid has a nucleotide sequence having at least 80%, 85%, 90%, 95%, 99%, or 100% sequence identity to SEQ ID NO: 51. In one specific embodiment, the recombinant nucleic acid has a nucleotide sequence having at least 80%, 85%, 90%, 95%, 99%, or 100% sequence identity to SEQ ID NO: 51.

In some further embodiments, the recombinant nucleic acid further includes an additional nucleic acid encoding a Cas protein and/or a fusion protein described herein. Accordingly, in some embodiments, the recombinant nucleic acid includes a first nucleotide sequence that produces a guide RNA as described herein and a second nucleotide sequence that encodes a Cas protein described herein. In some embodiments, the recombinant nucleic acid includes a first nucleotide sequence that produces a guide RNA as described herein and a second nucleotide sequence that encodes a fusion protein described herein. In some embodiments, the recombinant nucleic acid includes a first nucleotide sequence that produces a guide RNA as described herein, a second nucleotide sequence encoding a Cas protein described herein, and a third nucleotide sequence that encodes a fusion protein described herein. In some embodiments, the recombinant nucleic acid includes a first nucleotide sequence that produces a guide RNA as described herein, and a second nucleotide sequence encoding a Cas protein described herein and a fusion protein described herein. In some embodiments, the nucleotide sequence encoding a Cas protein described herein and a fusion protein described herein are present in the same nucleic acid molecule, whereas the nucleotide sequence producing the guide RNA is present on a separate nucleic acid molecule. In some embodiments, the nucleotide sequence encoding a Cas protein described herein and a fusion protein described herein and nucleotide sequence producing the guide RNA are present on the same nucleic acid molecule. In some embodiments, the nucleotide sequence encoding a Cas protein described herein and the nucleotide sequence encoding a fusion protein described herein are in the same open reading frame.

The present disclosure further provides an expression vector containing the recombinant nucleic acid as described herein. The expression vector can facilitate the expression of the recombinant nucleic acid in a prokaryotic, yeast, insect, or mammalian cell, or in vitro such that the composition encoded is produced, or into a subject in need thereof.

In some embodiments, the expression vector is designed for producing the payload encoded by the recombinant nucleic acid in a prokaryotic, yeast, insect, or mammalian cell, or in vitro. For example, the expression vector can be introduced into in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase. In some embodiments, the expression vectors can be introduced and propagated in a prokaryote. In some embodiments, a prokaryote is used to amplify copies of the expression vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith et al, Gene, 67:31-40 (1988)), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTRC (Amann et al., Gene, 69 (2): 301-315 (1988)) and pET-11d (Studier et al., GENE EXPRESSION TECHNOLOGY: METHODS IN ENZYMOLOGY 185, Academic Press, San Diego, Calif. (1990) 60-89).

In some embodiments, the expression vector is a yeast expression vector. Non-limiting examples of vectors for expression in yeast Saccharomyces cerevisae include pYepSecl (Baldari, et al., EMBO J., 6:229-234 (1987)), pMFa (Kurjan et al., Cell, 30:933-943 (1982)), pJRY88 (Schultz et al., Gene, 54:113-123 (1987)), pYES2 (Invitrogen Corporation, San Diego, Calif.), and picZ (Invitrogen Corp, San Diego, Calif.).

In some embodiments, the expression vector drives protein expression in insect cells using baculovirus expression vectors. Baculovirus vectors available for expression of proteins in cultured insect cells (e.g., SF9 cells) include the pAc series (Smith et al., Mol. Cell. Biol., 3 (12): 2156-2165 (1983)) and the pVL series (Lucklow et al., Virology, 170:31-39 (1989)).

In some embodiments, the expression vector is capable of driving expression of one or more sequences in mammalian cells using a mammalian expression vector. Examples of mammalian expression vectors include pCDM8 (Seed, Nature, 329:840 (1987)) and pMT2PC (Kaufman et al., EMBO J., 6:187-195 (1987)). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev., 1:268-277 (1987)), lymphoid-specific promoters (Calame et al., Adv. Immunol., 43:235-275 (1988)), in particular promoters of T cell receptors (Winoto et al., EMBO J., 8:729-733 (1989)) and immunoglobulins (Banciji et al., Cell, 33:729-740 (1983); Queen et al., Cell, 33:741-748 (1983)), neuron-specific promoters (e.g., the neurofilament promoter; Byrne et al., Proc. Natl. Acad. Sci. USA, 86:5473-5477 (1989)), pancreas-specific promoters (Edlund et al., Science, 230:912-916 (1985)), and mammary gland-specific promoters (e.g., milk whey promoter; U.S. Pat. No. 4,873,316 and European Publication No. 264,166). Developmentally-regulated promoters are also encompassed, e.g., the murine hox promoters (Kessel et al., Science, 249:374-379 (1990)) and the α-fetoprotein promoter (Campes et al., Genes Dev., 3:537-546 (1989)).

### Composition Including the CRISPR-Based System

Some aspects of the present disclosure further provide a composition containing the CRISPR-based system described herein. For example, the composition can include a guide RNA, or a recombinant nucleic acid or expression vector producing the guide RNA. In some embodiments, the composition further includes a Cas protein, or a recombinant nucleic acid or expression vector encoding the Cas protein. In some other embodiments, the composition further includes a fusion protein described herein, or a recombinant nucleic acid or expression vector encoding the fusion protein.

In some embodiments, a composition includes (a) a guide RNA, or a recombinant nucleic acid or expression vector producing the guide RNA; (b) a Cas protein, or a recombinant nucleic acid or expression vector encoding the Cas protein; and (c) a fusion protein or a recombinant nucleic acid or expression vector encoding the fusion protein.

In some specific embodiments, the composition includes a guide RNA, a Cas protein, and a fusion protein having a T4 DNA polymerase segment and a segment of an MS2 bacteriophage coat protein. In some specific embodiments, the composition includes a guide RNA, a Cas9 protein, and a fusion protein having a T4 DNA polymerase segment and a segment of an MS2 bacteriophage coat protein.

In some embodiments, the composition includes a cell introduced with the CRISPR-based system described herein. Accordingly, in some embodiments, at least one genomic locus of the cell is modified such that the expression the gene encoded by the modified genomic locus is decreased. The cell carrying the genomic locus modification can be introduced into a subject in need thereof.

In some embodiments, the recombinant nucleic acid can be introduced into a cell by methods and technologies known in the art. For example, a recombinant nucleic acid can be introduced into a cell using a viral vector. Non-limiting examples of viral vectors include adenovirus vectors, adeno-associated virus vectors, retrovirus vectors, lentivirus vectors, Sendai virus vectors, and the like.

Methods of non-viral delivery or introduction of nucleic acids into cells include lipofection, nucleofection, microinjection, biolistics, virosomes, liposomes, immunoliposomes, polycation or lipid-nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g., in vitro or ex vivo administration) or target tissues (e.g., in vivo administration). The preparation of lipid-nucleic acid complexes, including targeted liposomes such as immunolipid complexes, is well known in the art (see, e.g., Crystal, Science, 270 (5235): 404-410 (1995); Blaese et al., Cancer Gene Ther., 2:291-297 (1995); Behr, Bioconjugate Chem., 5:382-389 (1994); Remy et al., Bioconjugate Chem., 5:647-654 (1994); Gao et al., Gene Therapy, 2 (10): 710-722 (1995); Ahmad et al., Cancer Res., 52:4817-4820 (1992); U.S. Pat. Nos. 4,186,183, 4,217,344, 4,235,871, 4,261,975, 4,485,054, 4,501,728, 4,774,085, 4,837,028, and 4,946,787). Methods for non-viral delivery or introduction of nucleic acids into cells are also exemplified by Example 1.

Some aspects of the present disclosure further provide a pharmaceutical composition containing the guide RNA as described herein. In some embodiments, a pharmaceutical composition further including the Cas protein and/or the fusion protein as described herein, or recombinant nucleic acid or the expression vector encoding the Cas protein and/or the fusion protein. In some embodiments, the pharmaceutical composition further includes a pharmaceutically acceptable carrier, diluent, and/or excipient described herein.

In some embodiments, the pharmaceutical composition includes a recombinant nucleic acid or the expression vector producing the guide RNA. In some embodiments, the recombinant nucleic acid or the expression vector further encodes the Cas protein and/or the fusion protein as described herein. In some embodiments, the pharmaceutical composition further includes the Cas protein and/or the fusion protein as described herein, or recombinant nucleic acid or the expression vector encoding the Cas protein and/or the fusion protein. In some embodiments, the pharmaceutical composition further includes a pharmaceutically acceptable carrier, diluent, and/or excipient described herein.

In some embodiments, the pharmaceutical composition includes cells introduced with the guide RNA, or a recombinant nucleic acid or expression vector producing the guide RNA, and/or the Cas protein and/or the fusion protein as described herein. In some embodiments, the pharmaceutical composition further includes the guide RNA, the Cas protein and/or the fusion protein as described herein. In some embodiments, the pharmaceutical composition further includes a pharmaceutically acceptable carrier, diluent, and/or excipient described herein.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated.

Examples of suitable aqueous and nonaqueous carriers which can be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it can be useful to include isotonic agents, for example, sugars, polyalcohols such as mannitol, sorbitol, or sodium chloride in the composition. Prolonged absorption of the injectable compositions can be brought about by including in the composition, an agent that delays absorption, for example, monostearate salts and gelatin.

The pharmaceutical compositions can also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration. Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) can be mixed under sterile conditions with additional pharmaceutically acceptable carrier(s), and with any preservatives, buffers, or propellants which may be required.

Prevention of presence of microorganisms can be ensured both by sterilization procedures, supra, and by the inclusion of various antibacterial and antifungal agents, for example, paraben, chlorobutanol phenol sorbic acid, and the like. it can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Another aspect of the disclosure provides a kit including one or more components described herein. For example, in some embodiments, the kit can include a guide RNA described herein. In some embodiments, the kit can contain a Cas protein, and/or a fusion protein described herein. In some embodiments, the kit can contain the system or composition that include the guide RNA, Cas protein, and/or a fusion protein described herein. In some embodiments, the kit contains one or more recombinant nucleic acid, or one or more expression vector as described herein. In yet another embodiment, the kit contains a pharmaceutical composition described herein. In some other embodiments, the kit contains the cell as described herein.

In some embodiments, the kit comprises one or more reagents for use in a process utilizing one or more of the components described herein. Reagents can be provided in any suitable container. For example, a kit can provide one or more reaction or storage buffers. Reagents can be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

In some embodiments, a kit can contain the composition as described herein. If the composition containing components for administration is not formulated for delivery via the alimentary canal, a device capable of delivering the kit components through some other route can be included. One type of device, for applications such as parenteral delivery, is a syringe that is used to inject the composition into the body of a subject. Inhalation devices can also be used. The kit components can be packaged together or separated into two or more containers. In some embodiments, the containers can be vials that contain sterile, lyophilized formulations of a composition that are suitable for reconstitution. A kit can also contain one or more buffers suitable for reconstitution and/or dilution of other reagents. Other containers that can be used include, but are not limited to, a pouch, tray, box, tube, or the like. Kit components can be packaged and maintained sterilely within the containers. Another component that can be included is instructions to a person using a kit for its use.

### III. Method of Treating Diseases

One aspect of the disclosure provides methods of treating diseases that benefit from modulation of one or more DNA mismatch repair genes. Non-limiting examples of DNA mismatch repair genes include MSH2, MSH3, MLH1, PMS2, and MLH3. Any other DNA mismatch repair genes known in the art can be modulated in the methods described herein. A disease benefiting from modulation of one or more DNA mismatch repair genes is a disease that is associated with higher or lower expression, or higher or lower activity of the one or more DNA mismatch repair genes than the levels in a healthy subject. Accordingly, in some embodiments, the disease can benefit from modulating the one or more DNA mismatch repair genes to decrease the expression or activity of the one or more DNA mismatch repair genes. In some other embodiments, the disease can benefit from modulating the one or more DNA mismatch repair genes to increase the expression or activity of the one or more DNA mismatch repair genes. In one particular embodiment, the disease benefits from modulation of MSH3 gene.

In some aspects, the disease that benefits from modulation of one or more DNA mismatch repair genes is a DNA repeat expansion-associated disease. Accordingly, in some embodiments, the disclosure provides methods of treating a DNA repeat expansion-associated disease in a subject in need thereof, including introducing a CRISPR-based composition or system described herein, or a recombinant nucleic acid or an expression vector as described herein, to a subject. A DNA repeat expansion can be located in exons or introns of a gene in a subject, and subsequently disrupt the normal function or activity of the gene. A type of DNA repeat expansion is trinucleotide repeat expansion, which, as described elsewhere in the disclosure, is a series of three bases (e.g., CAG) repeated at least twice. Certain DNA repeat expansions are associated with a number of diseases, including but not limited to myotonic dystrophy type 1 (DM1), Huntington's disease (HD), fragile-X related disorders (FXDs), fragile XE MR (FRAXE), Freidreich ataxia (FRDA), spinal and bulbar muscular atrophy (SBMA), spinocerebellar ataxia type 1 (SCA1), spinocerebellar ataxia type 8 (SCA8), and spinocerebellar ataxia type 12 (SCA12).

Certain mismatch repair genes (e.g., MSH2, MSH3, MLH1, PMS2, and MLH3) modify disease onset by promoting repeat expansion in a tissue-specific manner. Thus, by targeting and decreasing the expression of a mismatch repair gene (e.g., MSH3) via introducing a composition, recombinant nucleic acid, or expression vector described herein, a DNA repeat expansion-associated disease can be treated. In some embodiments, the introduction of the composition, the recombinant nucleic acid or the expression vector introduces a frameshift mutation in an exon of a mismatch repair gene in a cell of the subject. In some other embodiments, a frameshift mutation can be introduced to the conjunction of an exon and an intron of a mismatch repair gene in a cell of the subject. As a result, the introduction of the composition, the recombinant nucleic acid or the expression vector decreases the expression level of the mismatch repair gene in a cell of the subject by at least 10%, 20%, 30%, 40%, 50%, 60%, 70%, 80%, 90%, 95%, 99%, or 100%. In some embodiments, the decrease in expression level of the mismatch repair gene in a cell is a decrease in the transcript levels of said mismatch repair gene. In some embodiments, the composition, recombinant nucleic acid, or expression vector described herein introduces less on-target large structural variants compared with the same composition, recombinant nucleic acid, or expression vector but without the fusion protein.

The subject in need thereof can be a subject who is diagnosed with one or more DNA-expansion associated diseases. Alternatively, the subject in need thereof may not yet show disease symptom but is determined to be at risk of developing one or more DNA-expansion associated diseases, as assessed by the presence of risk factors and/or assessment method described herein or known in the art. Non-limiting examples of risk factors for DNA-expansion associated diseases include high DNA repeat numbers at certain genomic loci compared to the reference number known in the art or from a healthy population; and hereditary history etc.

The composition decreases the expression of at least one mismatch repair genes (e.g., MSH2, MSH3, MLH1, PMS2, and MLH3), which modify disease onset by promoting repeat expansion in a tissue-specific manner. Thus, the composition stops DNA expansion of at least one genomic loci of the subject, stops at least one DNA expansion-associated disease progression, and/or prevents at least one DNA expansion-associated disease onset. In some embodiments, the subject has not yet shown disease onset but is at risk of developing at least one DNA expansion-associated disease. Accordingly, in some embodiments, the composition prevents at least one DNA expansion-associated disease onset and/or lowers the risk of the same. In some other embodiments, the subject has at least one DNA expansion-associated disease onset. Accordingly, the composition stops disease progression and improves disease symptoms.

In some embodiments, the subject in need thereof is a human subject. In some specific embodiments, the human subject is an adult. In some embodiments, the introduction of the composition, the recombinant nucleic acid or the expression vector introduces a frameshift mutation to an exon of MSH3 in a cell of the subject. In some other embodiments, the frameshift mutation can be introduced to a conjunction of an exon and an intron of MSH3 in a cell of the subject. In some specific embodiments, the frameshift mutation can be introduced to any one of exons 1-6 of MSH3 in a cell of the subject. In one embodiment, the introduction of the composition, the recombinant nucleic acid or the expression vector decreases the expression level of MSH3. In some embodiments, the decrease in expression level of the MSH3 in a cell is a decrease in the transcript levels of MSH3 in the cell. In some embodiments, the composition halts DNA expansion of at least one genomic loci of the subject.

MSH3, also known as MutS Homolog 3, DUP, FAP4, and MRP1, includes a protein that forms a heterodimer with MSH2 to form MutS beta, part of the post-replicative DNA mismatch repair system. Defects in MSH3 are known to cause susceptibility to endometrial cancer (Tseng-Rogenski et al., Mol Cell Biol, 40 (13): e00029-20 (2020)).

The encoding nucleotide sequence of Homo sapiens MSH3 (Genebank ID: 4437) has the nucleotide sequence of:

(SEQ ID NO: 75)
ATGTCTCGCCGGAAGCCTGCGTCGGGCGGCCTCGCTGCCTCCAGCTCAG
CCCCTGCGAGGCAAGCGGTTTTGAGCCGATTCTTCCAGTCTACGGGAAG
CCTGAAATCCACCTCCTCCTCCACAGGTGCAGCCGACCAGGTGGACCCT
GGCGCTGCAGCGGCTGCAGCGGCCGCAGCGGCCGCAGCGCCCCCAGCGC
CCCCAGCTCCCGCCTTCCCGCCCCAGCTGCCGCCGCACATAGCTACAGA
AATTGACAGAAGAAAGAAGAGACCATTGGAAAATGATGGGCCTGTTAAA
AAGAAAGTAAAGAAAGTCCAACAAAAGGAAGGAGGAAGTGATCTGGGAA
TGTCTGGCAACTCTGAGCCAAAGAAATGTCTGAGGACCAGGAATGTTTC
AAAGTCTCTGGAAAAATTGAAAGAATTCTGCTGCGATTCTGCCCTTCCT
CAAAGTAGAGTCCAGACAGAATCTCTGCAGGAGAGATTTGCAGTTCTGC
CAAAATGTACTGATTTTGATGATATCAGTCTTCTACACGCAAAGAATGC
AGTTTCTTCTGAAGATTCGAAACGTCAAATTAATCAAAAGGACACAACA
CTTTTTGATCTCAGTCAGTTTGGATCATCAAATACAAGTCATGAAAATT
TACAGAAAACTGCTTCCAAATCAGCTAACAAACGGTCCAAAAGCATCTA
TACGCCGCTAGAATTACAATACATAGAAATGAAGCAGCAGCACAAAGAT
GCAGTTTTGTGTGTGGAATGTGGATATAAGTATAGATTCTTTGGGGAAG
ATGCAGAGATTGCAGCCCGAGAGCTCAATATTTATTGCCATTTAGATCA
CAACTTTATGACAGCAAGTATACCTACTCACAGACTGTTTGTTCATGTA
CGCCGCCTGGTGGCAAAAGGATATAAGGTGGGAGTTGTGAAGCAAACTG
AAACTGCAGCATTAAAGGCCATTGGAGACAACAGAAGTTCACTCTTTTC
CCGGAAATTGACTGCCCTTTATACAAAATCTACACTTATTGGAGAAGAT
GTGAATCCCCTAATCAAGCTGGATGATGCTGTAAATGTTGATGAGATAA
TGACTGATACTTCTACCAGCTATCTTCTGTGCATCTCTGAAAATAAGGA
AAATGTTAGGGACAAAAAAAAGGGCAACATTTTTATTGGCATTGTGGGA
GTGCAGCCTGCCACAGGCGAGGTTGTGTTTGATAGTTTCCAGGACTCTG
CTTCTCGTTCAGAGCTAGAAACCCGGATGTCAAGCCTGCAGCCAGTAGA
GCTGCTGCTTCCTTCGGCCTTGTCCGAGCAAACAGAGGCGCTCATCCAC
AGAGCCACATCTGTTAGTGTGCAGGATGACAGAATTCGAGTCGAAAGGA
TGGATAACATTTATTTTGAATACAGCCATGCTTTCCAGGCAGTTACAGA
GTTTTATGCAAAAGATACAGTTGACATCAAAGGTTCTCAAATTATTTCT
GGCATTGTTAACTTAGAGAAGCCTGTGATTTGCTCTTTGGCTGCCATCA
TAAAATACCTCAAAGAATTCAACTTGGAAAAGATGCTCTCCAAACCTGA
GAATTTTAAACAGCTATCAAGTAAAATGGAATTTATGACAATTAATGGA
ACAACATTAAGGAATCTGGAAATCCTACAGAATCAGACTGATATGAAAA
CCAAAGGAAGTTTGCTGTGGGTTTTAGACCACACTAAAACTTCATTTGG
GAGACGGAAGTTAAAGAAGTGGGTGACCCAGCCACTCCTTAAATTAAGG
GAAATAAATGCCCGGCTTGATGCTGTATCGGAAGTTCTCCATTCAGAAT
CTAGTGTGTTTGGTCAGATAGAAAATCATCTACGTAAATTGCCCGACAT
AGAGAGGGGACTCTGTAGCATTTATCACAAAAAATGTTCTACCCAAGAG
TTCTTCTTGATTGTCAAAACTTTATATCACCTAAAGTCAGAATTTCAAG
CAATAATACCTGCTGTTAATTCCCACATTCAGTCAGACTTGCTCCGGAC
CGTTATTTTAGAAATTCCTGAACTCCTCAGTCCAGTGGAGCATTACTTA
AAGATACTCAATGAACAAGCTGCCAAAGTTGGGGATAAAACTGAATTAT
TTAAAGACCTTTCTGACTTCCCTTTAATAAAAAAGAGGAAGGATGAAAT
TCAAGGTGTTATTGACGAGATCCGAATGCATTTGCAAGAAATACGAAAA
ATACTAAAAAATCCTTCTGCACAATATGTGACAGTATCAGGACAGGAGT
TTATGATAGAAATAAAGAACTCTGCTGTATCTTGTATACCAACTGATTG
GGTAAAGGTTGGAAGCACAAAAGCTGTGAGCCGCTTTCACTCTCCTTTT
ATTGTAGAAAATTACAGACATCTGAATCAGCTCCGGGAGCAGCTAGTCC
TTGACTGCAGTGCTGAATGGCTTGATTTTCTAGAGAAATTCAGTGAACA
TTATCACTCCTTGTGTAAAGCAGTGCATCACCTAGCAACTGTTGACTGC
ATTTTCTCCCTGGCCAAGGTCGCTAAGCAAGGAGATTACTGCAGACCAA
CTGTACAAGAAGAAAGAAAAATTGTAATAAAAAATGGAAGGCACCCTGT
GATTGATGTGTTGCTGGGAGAACAGGATCAATATGTCCCAAATAATACA
GATTTATCAGAGGACTCAGAGAGAGTAATGATAATTACCGGACCAAACA
TGGGTGGAAAGAGCTCCTACATAAAACAAGTTGCATTGATTACCATCAT
GGCTCAGATTGGCTCCTATGTTCCTGCAGAAGAAGCGACAATTGGGATT
GTGGATGGCATTTTCACAAGGATGGGTGCTGCAGACAATATATATAAAG
GACAGAGTACATTTATGGAAGAACTGACTGACACAGCAGAAATAATCAG
AAAAGCAACATCACAGTCCTTGGTTATCTTGGATGAACTAGGAAGAGGG
ACGAGCACTCATGATGGAATTGCCATTGCCTATGCTACACTTGAGTATT
TCATCAGAGATGTGAAATCCTTAACCCTGTTTGTCACCCATTATCCGCC
AGTTTGTGAACTAGAAAAAAATTACTCACACCAGGTGGGGAATTACCAC
ATGGGATTCTTGGTCAGTGAGGATGAAAGCAAACTGGATCCAGGCGCAG
CAGAACAAGTCCCTGATTTTGTCACCTTCCTTTACCAAATAACTAGAGG
AATTGCAGCAAGGAGTTATGGATTAAATGTGGCTAAACTAGCAGATGTT
CCTGGAGAAATTTTGAAGAAAGCAGCTCACAAGTCAAAAGAGCTGGAAG
GATTAATAAATACGAAAAGAAAGAGACTCAAGTATTTTGCAAAGTTATG
GACGATGCATAATGCACAAGACCTGCAGAAGTGGACAGAGGAGTTCAAC
ATGGAAGAAACACAGACTTCTCTTCTTCATTAA

In certain embodiments, the recombinant nucleic acid or the expression vector does not change the expression of DHFR gene compared with a control where the recombinant nucleic acid or the expression vector is not used, or where a control nucleic acid or a control expression vector (e.g., having the guide sequence of SEQ ID NO: 16 or 23) is used.

DHFR, as known as dihydrofolate reductase, DHFR1, DYR and DHFRP1, includes a protein that is a member of the reductase family of enzymes, which is ubiquitously expressed in all organisms. DHFR converts dihydrofolate into tetrahydrofolate, a methyl group shuttle required for the de novo synthesis of purines, thymidylic acid, and certain amino acids. Dihydrofolate reductase deficiency has been linked to megaloblastic anemia. DHFR has a key role in cell growth and proliferation.

The encoding nucleotide sequence of Homo sapiens DHFR (GeneBank ID: 1719) has the sequence of:

(SEQ ID NO: 76)
ATGGTTGGTTCGCTAAACTGCATCGTCGCTGTGTCCCAGAACATGGGCA
TCGGCAAGAACGGGGACCTGCCCTGGCCACCGCTCAGGAATGAATTCAG
ATATTTCCAGAGAATGACCACAACCTCTTCAGTAGAAGGTAAACAGAAT
CTGGTGATTATGGGTAAGAAGACCTGGTTCTCCATTCCTGAGAAGAATC
GACCTTTAAAGGGTAGAATTAATTTAGTTCTCAGCAGAGAACTCAAGGA
ACCTCCACAAGGAGCTCATTTTCTTTCCAGAAGTCTAGATGATGCCTTA
AAACTTACTGAACAACCAGAATTAGCAAATAAAGTAGACATGGTCTGGA
TAGTTGGTGGCAGTTCTGTTTATAAGGAAGCCATGAATCACCCAGGCCA
TCTTAAACTATTTGTGACAAGGATCATGCAAGACTTTGAAAGTGACACG
TTTTTTCCAGAAATTGATTTGGAGAAATATAAACTTCTGCCAGAATACC
CAGGTGTTCTCTCTGATGTCCAGGAGGAGAAAGGCATTAAGTACAAATT
TGAAGTATATGAGAAGAATGATTAA

A number of DNA expansion-associated diseases can be treated by the method described herein.

Myotonic dystrophy type 1, also known as Steinert's disease, is a multisystem genetic disorder that affects skeletal and smooth muscle as well as the eye, heart, endocrine system, and central nervous system. The clinical findings, which span a continuum from mild to severe, have been categorized into three somewhat overlapping phenotypes: mild, classic, and congenital. Mild DM1 is characterized by cataract and mild myotonia (sustained muscle contraction); life span is normal. Classic DM1 is characterized by muscle weakness and wasting, myotonia, cataract, and often cardiac conduction abnormalities; adults can become physically disabled and can have a shortened life span. Congenital DM1 is characterized by hypotonia and severe generalized weakness at birth, often with respiratory insufficiency and early death; intellectual disability is common. DM1 is caused by expansion of a CTG trinucleotide repeat in the noncoding region of DMPK. DM1 is inherited in an autosomal dominant manner. Pathogenic alleles can expand in length during gametogenesis, resulting in the transmission of longer trinucleotide repeat alleles that can be associated with earlier onset and more severe disease than that observed in the parent. The diagnosis of DM1 is suspected in individuals with characteristic muscle weakness and is confirmed by molecular genetic testing of DMPK. CTG repeat length exceeding 34 repeats is abnormal. Molecular genetic testing detects pathogenic variants in nearly 100% of affected individuals.

Huntington's disease is a neurodegenerative disorder of the central nervous system characterized by unwanted choreatic movements, behavioral and psychiatric disturbances and dementia. About eight percent of cases start before the age of 20 years, and are known as juvenile HD, which typically present with the slow movement symptoms of Parkinson's disease rather than those of chorea. HD is typically inherited from an affected parent, who carries a mutation in the huntingtin gene (HTT), which encodes huntingtin (Htt). Htt is expressed in all cells, with the highest concentrations found in the brain and testes, and moderate amounts in the liver, heart, and lungs. The functions of Htt are not clear, it is known that Htt interacts with proteins involved in transcription, cell signaling, and intracellular transporting. Expansion of CAG repeats in HTT gene results in an abnormal mutant Htt, which gradually damages brain cells through a number of possible mechanisms.

Fragile-X related disorders (FXDs), also known as, includes Fragile X syndrome (FXS), Fragile X-associated primary ovarian insufficiency (FXPOI) and Fragile X-associated tremor/ataxia syndrome (FXTAS). Fragile X syndrome occurs in individuals with an FMRI full mutation or other loss-of-function variant and is nearly always characterized in affected males by developmental delay and intellectual disability along with a variety of behavioral issues. FXTAS occurs in individuals who have an FMRI premutation and is characterized by late-onset, progressive cerebellar ataxia and intention tremor followed by cognitive impairment. Psychiatric disorders are common. Age of onset is typically between 60 and 65 years and is more common among males who are hemizygous for the premutation (40%) than among females who are heterozygous for the premutation (16%-20%). FXPOI, defined as hypergonadotropic hypogonadism before age 40 years, has been observed in 20% of women who carry a premutation allele compared to 1% in the general population. The diagnosis of an FMRI disorder is established through the use of specialized molecular genetic testing to detect CGG trinucleotide repeat expansion in the 5′ UTR of FMRI with abnormal gene methylation for most alleles with >200 repeats. Typically, a definite diagnosis of FXS requires the presence of a full-mutation repeat size (>200 CGG repeats (SEQ ID NO: 86)) while the diagnosis of FXTAS or FXPOI is associated with a premutation-sized repeat (55-200 CGG repeats (SEQ ID NO: 87)).

Fragile XE syndrome (FRAXE) is a genetic disorder characterized by mildly to moderately impaired intellectual development associated with learning difficulties, communication deficits, attention problems, hyperactivity, and autistic behavior. FRAXE is caused by the silencing of AFF2, which is an X-linked gene expressed in primarily in placenta and adult and fetal brain. AFF2 is silenced in a mild to borderline form of non-syndromic intellectual disability (ID), the FRAXE mental retardation due to a CCG expansion (>200 repeats (SEQ ID NO: 88)) located in the 5′-untranslated region of this gene, resulting in the FRAXE fragile site and the lack of AFF2 protein.

Freidreich ataxia (FRDA) is an autosomal-recessive genetic disease that causes difficulty walking, a loss of coordination in the arms and legs, and impaired speech that worsens over time. Symptoms generally start between 5 and 20 years of age. Many develop hypertrophic cardiomyopathy and require a mobility aid such as a cane, walker, or wheelchair in their teens. As the disease progresses, some affected people lose their sight and hearing. Other complications can include scoliosis and diabetes mellitus. FRDA is caused by mutations and silencing of FXN gene. In most cases, the mutant FXN gene has 90-1,300 GAA trinucleotide repeat (SEQ ID NO: 89) expansions in intron 1 of both alleles, causing epigenetic changes and formation of heterochromatin near the repeat, and subsequent silencing of the FXN gene.

Spinal and bulbar muscular atrophy (SBMA) is a gradually progressive neuromuscular disorder in which degeneration of lower motor neurons results in muscle weakness, muscle atrophy, and fasciculations in affected males. Affected individuals often show gynecomastia, testicular atrophy, and reduced fertility as a result of mild androgen insensitivity. The diagnosis of SBMA is established in a male proband by the identification of a hemizygous expansion of a CAG trinucleotide repeat (>35 CAGs (SEQ ID NO: 90)) in AR by molecular genetic testing. For management of SBMA, use of braces and walkers for ambulation as needed as the disease progresses; standard treatments for dysarthria and dysphagia; breast reduction surgery for gynecomastia as needed; standard treatment per cardiologist and/or endocrinologist for cardiac manifestations and metabolic syndrome; psychosocial support and education to decrease stress and burden on caregivers.

Spinocerebellar ataxia type 1 (SCA1), also known as Spinocerebellar atrophy I, is characterized by progressive cerebellar ataxia, dysarthria, and eventual deterioration of bulbar functions. Early in the disease, affected individuals may have gait disturbance, slurred speech, difficulty with balance, brisk deep tendon reflexes, hypermetric saccades, nystagmus, and mild dysphagia. Later signs include slowing of saccadic velocity, development of upgaze palsy, dysmetria, dysdiadochokinesia, and hypotonia. In advanced stages, muscle atrophy, decreased deep tendon reflexes, loss of proprioception, cognitive impairment (e.g., frontal executive dysfunction, impaired verbal memory), chorea, dystonia, and bulbar dysfunction are seen. Onset is typically in the third or fourth decade, although childhood onset and late-adult onset have been reported. Those with onset after age 60 years may manifest a pure cerebellar phenotype. Interval from onset to death varies from ten to 30 years; individuals with juvenile onset show more rapid progression and more severe disease. Anticipation is observed. An axonal sensory neuropathy detected by electrophysiologic testing is common; brain imaging typically shows cerebellar and brain stem atrophy.

Mutations in the ATXN1 gene, which encodes ataxin-1, cause SCA1. Ataxin-1 is a nuclear protein found throughout the body. It is believed that ataxin-1 is involved in regulating various aspects of producing proteins, including the transcription and RNA processing.

The ATXN1 gene mutations that cause SCA1 are associated with a CAG trinucleotide repeat expansion. Normally, the CAG segment is repeated 4 to 39 times (SEQ ID NO: 91) within the ATXN1 gene. In the case of trinucleotide repeat expansion, the CAG segment is repeated 40 to more than 80 times (SEQ ID NO: 92). People with 40 to 50 repeats tend to first experience signs and symptoms of SCA1 in mid-adulthood, while people with more than 70 repeats usually have signs and symptoms by their teens. SCA1 is inherited in an autosomal dominant pattern. An affected person usually inherits the altered gene from one affected parent. However, some people with SCA1 do not have a parent with the disorder.

The diagnosis of SCA1 is established in a proband with characteristic clinical findings and an abnormal CAG repeat expansion in ATXN1 identified by molecular genetic testing. Affected individuals usually have 39 or more CAG repeats (SEQ ID NO: 93).

For management of SCA1, supportive care including adaptive devices, physical therapy, occupational therapy, avoidance of obesity; intensive rehabilitation (coordinative physiotherapy) can be beneficial; speech therapy and communication devices for dysarthria; video esophagram to help identify the consistency of food least likely to trigger aspiration and feeding devices can be indicated with recurrent aspiration; caloric support for those with weight loss; vitamin supplementation as needed; psychotherapy, neuropsychologic rehabilitation, and/or standard psychiatric treatments for cognitive and psychiatric manifestations; pharmacotherapy and/or referral to pain management as needed for pain.

Spinocerebellar ataxia type 8 (SCA8) is an inherited neurodegenerative characterized by a slowly progressive ataxia with onset typically in the third to fifth decade but with a range from before age one year to after age 60 years. Common initial manifestations are scanning dysarthria with a characteristic drawn-out slowness of speech and gait instability. Over the disease course other findings can include eye movement abnormalities (nystagmus, abnormal pursuit and abnormal saccades, and, rarely, ophthalmoplegia); upper motor neuron involvement; extrapyramidal signs; brain stem signs (dysphagia and poor cough reflex); sensory neuropathy; and cognitive impairment (e.g., executive dysfunction, psychomotor slowing and other features of cerebellar cognitive-affective disorder in some). The diagnosis of SCA8 is established in a proband with suggestive findings and a heterozygous abnormal CTG⋅CAG repeat expansion in the two overlapping genes ATXN8OS/ATXN8. 54-250 CTG⋅CAG repeats are most often seen in individuals with ataxia.

Spinocerebellar ataxia type 12 (SCA12) is a late-onset, autosomal dominant, slowly progressive disorder characterized by an action tremor. The constellation of clinical signs and symptoms may vary within and among families with SCA12. Most patients with SCA12 display action tremor of various body parts in the third to fifth decade and later develop hyperreflexia and gait ataxia along with other signs of cerebellar dysfunction, including limb dysmetria, dysarthria, and abnormal eye movements. In addition, some patients exhibit mild parkinsonism, anxiety, and mood disorders. In later stages, patients with SCA12 may develop dementia and autonomic dysfunction. SCA12 is caused by a CAG repeat expansion mutation in exon 7 of PPP2R2B, a gene that encodes a regulatory subunit of protein phosphatase 2A (PP2A). CAG repeats number 7-28 in normal individuals (SEQ ID NO: 94) and 55-78 in SCA12 patients (SEQ ID NO: 95).

The pharmaceutical composition can be introduced to the subject in need thereof via various method known in the art. For example, the pharmaceutical composition can be introduced to the subject by parenteral administration, meaning means modes of administration other than enteral and topical administration, usually by injection. Non-limiting examples of parenteral administration include, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Other method of introduction includes enteral, and topical administration.

Generally, the dosage of administering the pharmaceutical composition described herein to human subjects varies depending on factors such as the subject's age, weight, height, sex, general medical condition, and previous medical history. The pharmaceutical composition may be administered at a dosage from about 0.1 mg/kg to about 100 mg/kg in a single dose. Non-limiting examples of dosages include, but are not limited to, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, 1 about 2 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg or more. A person skilled in the art can determine the administering dosage based the afore-mentioned factors. Additional non-limiting examples of the factors to consider include family disease history, and increased DNA repeat number at certain genetic loci as compared to a proper reference number such as the average DNA repeat number at the same genetic loci from a healthy population.

The dosage can be administered multiple times, as needed. The dosage can be administered once, twice or three times a week, at a schedule of weekly, every other week, one week of administration followed by two, three or four weeks off, two weeks of administration followed by one, two, three or four weeks off, three weeks of administration followed by one, two, three, four or five week off, four weeks of administration followed by one, two, three, four or five week off, five weeks of administration followed by one, two, three, four or five week off, or monthly. The administration schedule can be repeated as many times as needed, depending on factors such as the disease condition etc. A person skilled in the art can adjust the dosage and dosage schedule based on the above-mentioned factors.

TABLE 1
TABLES AND SEQUENCES
Exemplary nucleotide sequences of hybridization sequences for guide RNAs

| Target Loci | | | SEQ | |
|---|---|---|---|---|
| of | | Target | ID | |
| Human MSH3 | Name | Strand | NO | RNA Sequence (5′→3′) |
| Exon 3 | Exon 3 SG 1 | + | 1 | CAAAGAAAUGUCUGAGGACC |
| Exon 4 | Exon 4 SG 1 | − | 2 | GGACCGUUUGUUAGCUGAUU |
| Exon 5 | Exon 5 SG 1 | + | 3 | GUUUGUUCAUGUACGCCGCC |
| Exon 5 | Exon 5 SG 2 | + | 4 | UGUUCAUGUACGCCGCCUGG |
| Exon 5 | Exon 5 SG 3 | + | 5 | GUACGCCGCCUGGUGGCAAA |
| Exon 6 | Exon 6 SG 1 | + | 6 | ACUGCAGCAUUAAAGGCCAU |
| Negative | LacZ | + | 7 | UGCGAAUACGCCCACGCGAU |
| controls | | | | |

TABLE 2
Exemplary nucleotide sequences of scaffold RNA for guide RNAs

| Name | SEQ ID NO | RNA Sequence (5′→3′) |
|---|---|---|
| MS2 Scaffold | 8 | GUUUUAGAGCUAGGCCAACAUGAGGAUCAC |
| | | CCAUGUCUGCAGGGCCUAGCAAGUUAAAAU |
| | | AAGGCUAGUCCGUUAUCAACUUGGCCAACAU |
| | | GAGGAUCACCCAUGUCUGCAGGGCCAAGUGG |
| | | CACCGAGUCGGUGCUUU |
| Cas9 Scaffold | 9 | GUUUUAGAGCUAGAAAUAGCAAGUUAAAAU |
| | | AAGGCUAGUCCGUUAUCAACUUGAAAAAGU |
| | | GGCACCGAGUCGGUGCUUU |

“MS2 Scaffold” = A scaffold containing both Cas9 binding motif and MS2 binding motifs
Underline: MS2 binding motif
“Cas9 Scaffold” = Cas9 binding sequence

TABLE 3
Exemplary nucleotide sequences of full guide RNAs (sgRNAs)

| Target | | | | |
|---|---|---|---|---|
| Loci of | | | SEQ | |
| Human | | Target | ID | |
| MSH3 | Name | Strand | NO | RNA Sequence (5′→3′) |
| Exon 3 | Exon 3 | + | 10 | CAAAGAAAUGUCUGAGGACCGUUUUAG |
| | SG 1 | | | AGCUAGGCCAACAUGAGGAUCACCCAU |
| | MS2 | | | GUCUGCAGGGCCUAGCAAGUUAAAAUA |
| | Scaffold | | | AGGCUAGUCCGUUAUCAACUUGGCCAA |
| | | | | CAUGAGGAUCACCCAUGUCUGCAGGGCC |
| | | | | AAGUGGCACCGAGUCGGUGCUUU |
| Exon 4 | Exon 4 | − | 11 | GGACCGUUUGUUAGCUGAUUGUUUUAG |
| | SG 1 | | | AGCUAGGCCAACAUGAGGAUCACCCAU |
| | MS2 | | | GUCUGCAGGGCCUAGCAAGUUAAAAUA |
| | Scaffold | | | AGGCUAGUCCGUUAUCAACUUGGCCAA |
| | | | | CAUGAGGAUCACCCAUGUCUGCAGGGCC |
| | | | | AAGUGGCACCGAGUCGGUGCUUU |
| Exon 5 | Exon 5 | + | 12 | GUUUGUUCAUGUACGCCGCCGUUUUAG |
| | SG 1 | | | AGCUAGGCCAACAUGAGGAUCACCCAU |
| | MS2 | | | GUCUGCAGGGCCUAGCAAGUUAAAAUA |
| | Scaffold | | | AGGCUAGUCCGUUAUCAACUUGGCCAA |
| | | | | CAUGAGGAUCACCCAUGUCUGCAGGGCC |
| | | | | AAGUGGCACCGAGUCGGUGCUUU |
| Exon 5 | Exon 5 | + | 13 | UGUUCAUGUACGCCGCCUGGGUUUUAG |
| | SG 2 | | | AGCUAGGCCAACAUGAGGAUCACCCAU |
| | MS2 | | | GUCUGCAGGGCCUAGCAAGUUAAAAUA |
| | Scaffold | | | AGGCUAGUCCGUUAUCAACUUGGCCAA |
| | | | | CAUGAGGAUCACCCAUGUCUGCAGGGCC |
| | | | | AAGUGGCACCGAGUCGGUGCUUU |
| Exon 5 | Exon 5 | + | 14 | GUACGCCGCCUGGUGGCAAAGUUUUAG |
| | SG 3 | | | AGCUAGGCCAACAUGAGGAUCACCCAU |
| | MS2 | | | GUCUGCAGGGCCUAGCAAGUUAAAAUA |
| | Scaffold | | | AGGCUAGUCCGUUAUCAACUUGGCCAA |
| | | | | CAUGAGGAUCACCCAUGUCUGCAGGGCC |
| | | | | AAGUGGCACCGAGUCGGUGCUUU |
| Exon 6 | Exon 6 | + | 15 | ACUGCAGCAUUAAAGGCCAUGUUUUAG |
| | SG 1 | | | AGCUAGGCCAACAUGAGGAUCACCCAU |
| | MS2 | | | GUCUGCAGGGCCUAGCAAGUUAAAAUA |
| | Scaffold | | | AGGCUAGUCCGUUAUCAACUUGGCCAA |
| | | | | CAUGAGGAUCACCCAUGUCUGCAGGGCC |
| | | | | AAGUGGCACCGAGUCGGUGCUUU |
| N/A | Negative | + | 16 | UGCGAAUACGCCCACGCGAUGUUUUAG |
| | controls | | | AGCUAGGCCAACAUGAGGAUCACCCAU |
| | LacZ | | | GUCUGCAGGGCCUAGCAAGUUAAAAUA |
| | MS2 | | | AGGCUAGUCCGUUAUCAACUUGGCCAA |
| | Scaffold | | | CAUGAGGAUCACCCAUGUCUGCAGGGCC |
| | | | | AAGUGGCACCGAGUCGGUGCUUU |
| Exon 3 | Exon 3 | + | 17 | CAAAGAAAUGUCUGAGGACCGUUUUAG |
| | SG 1 | | | AGCUAGAAAUAGCAAGUUAAAAUAAGG |
| | Cas9 | | | CUAGUCCGUUAUCAACUUGAAAAAGUG |
| | Scaffold | | | GCACCGAGUCGGUGCUUU |
| Exon 4 | Exon 4 | − | 18 | GGACCGUUUGUUAGCUGAUUGUUUUAG |
| | SG 1 | | | AGCUAGAAAUAGCAAGUUAAAAUAAGG |
| | Cas9 | | | CUAGUCCGUUAUCAACUUGAAAAAGUG |
| | Scaffold | | | GCACCGAGUCGGUGCUUU |
| Exon 5 | Exon 5 | + | 19 | GUUUGUUCAUGUACGCCGCCGUUUUAG |
| | SG 1 | | | AGCUAGAAAUAGCAAGUUAAAAUAAGG |
| | Cas9 | | | CUAGUCCGUUAUCAACUUGAAAAAGUG |
| | Scaffold | | | GCACCGAGUCGGUGCUUU |
| Exon 5 | Exon 5 | + | 20 | UGUUCAUGUACGCCGCCUGGGUUUUAG |
| | SG 2 | | | AGCUAGAAAUAGCAAGUUAAAAUAAGG |
| | Cas9 | | | CUAGUCCGUUAUCAACUUGAAAAAGUG |
| | Scaffold | | | GCACCGAGUCGGUGCUUU |
| Exon 5 | Exon 5 | + | 21 | GUACGCCGCCUGGUGGCAAAGUUUUAG |
| | SG 3 | | | AGCUAGAAAUAGCAAGUUAAAAUAAGG |
| | Cas9 | | | CUAGUCCGUUAUCAACUUGAAAAAGUG |
| | Scaffold | | | GCACCGAGUCGGUGCUUU |
| Exon 6 | Exon 6 | + | 22 | ACUGCAGCAUUAAAGGCCAUGUUUUAG |
| | SG 1 | | | AGCUAGAAAUAGCAAGUUAAAAUAAGG |
| | Cas9 | | | CUAGUCCGUUAUCAACUUGAAAAAGUG |
| | Scaffold | | | GCACCGAGUCGGUGCUUU |
| N/A | Negative | + | 23 | UGCGAAUACGCCCACGCGAUGUUUUAG |
| | controls | | | AGCUAGAAAUAGCAAGUUAAAAUAAGG |
| | LacZ | | | CUAGUCCGUUAUCAACUUGAAAAAGUG |
| | Cas9 | | | GCACCGAGUCGGUGCUUU |
| | Scaffold | | | |

Underlined: hybridization sequence; UPPER CASE only: scaffold RNA.
“MS2 Scaffold” = Scaffold containing both Cas9 binding motif and MS2 binding motif
“Cas9 Scaffold” = Cas9 binding sequence

TABLE 4
Exemplary DNA sequences that encode full guide RNAs (sgRNAs)

| Target Loci | | | SEQ | |
|---|---|---|---|---|
| of Human | | Target | ID | |
| MSH3 | Name | Strand | NO | Full DNA sequence (5′→3′)* |
| Exon 3 | Exon 3 | + | 24 | CAAAGAAATGTCTGAGGACCGTTTTAGA |
| | SG 1 MS2 | | | GCTAGGCCAACATGAGGATCACCCATGT |
| | Scaffold | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| Exon 4 | Exon 4 | − | 25 | GGACCGTTTGTTAGCTGATTGTTTTAGAG |
| | SG 1 MS2 | | | CTAGGCCAACATGAGGATCACCCATGTC |
| | Scaffold | | | TGCAGGGCCTAGCAAGTTAAAATAAGGC |
| | | | | TAGTCCGTTATCAACTTGGCCAACATGAG |
| | | | | GATCACCCATGTCTGCAGGGCCAAGTGG |
| | | | | CACCGAGTCGGTGCTTT |
| Exon 5 | Exon 5 | + | 26 | GTTTGTTCATGTACGCCGCCGTTTTAGAG |
| | SG 1 MS2 | | | CTAGGCCAACATGAGGATCACCCATGTC |
| | Scaffold | | | TGCAGGGCCTAGCAAGTTAAAATAAGGC |
| | | | | TAGTCCGTTATCAACTTGGCCAACATGAG |
| | | | | GATCACCCATGTCTGCAGGGCCAAGTGG |
| | | | | CACCGAGTCGGTGCTTT |
| Exon 5 | Exon 5 | + | 27 | TGTTCATGTACGCCGCCTGGGTTTTAGAG |
| | SG 2 MS2 | | | CTAGGCCAACATGAGGATCACCCATGTC |
| | Scaffold | | | TGCAGGGCCTAGCAAGTTAAAATAAGGC |
| | | | | TAGTCCGTTATCAACTTGGCCAACATGAG |
| | | | | GATCACCCATGTCTGCAGGGCCAAGTGG |
| | | | | CACCGAGTCGGTGCTTT |
| Exon 5 | Exon 5 | + | 28 | GTACGCCGCCTGGTGGCAAAGTTTTAGA |
| | SG 3 MS2 | | | GCTAGGCCAACATGAGGATCACCCATGT |
| | Scaffold | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| Exon 6 | Exon 6 | + | 29 | ACTGCAGCATTAAAGGCCATGTTTTAGA |
| | SG 1 MS2 | | | GCTAGGCCAACATGAGGATCACCCATGT |
| | Scaffold | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| N/A | Negative | + | 30 | TGCGAATACGCCCACGCGATGTTTTAGA |
| | controls | | | GCTAGGCCAACATGAGGATCACCCATGT |
| | LacZ MS2 | | | CTGCAGGGCCTAGCAAGTTAAAATAAGG |
| | Scaffold | | | CTAGTCCGTTATCAACTTGGCCAACATGA |
| | | | | GGATCACCCATGTCTGCAGGGCCAAGTG |
| | | | | GCACCGAGTCGGTGCTTT |
| Exon 3 | Exon 3 | + | 31 | CAAAGAAATGTCTGAGGACCGTTTTAGA |
| | SG 1 Cas9 | | | GCTAGAAATAGCAAGTTAAAATAAGGCT |
| | Scaffold | | | AGTCCGTTATCAACTTGAAAAAGTGGCA |
| | | | | CCGAGTCGGTGCTTT |
| Exon 4 | Exon 4 | − | 32 | GGACCGTTTGTTAGCTGATTGTTTTAGAG |
| | SG 1 Cas9 | | | CTAGAAATAGCAAGTTAAAATAAGGCTA |
| | Scaffold | | | GTCCGTTATCAACTTGAAAAAGTGGCAC |
| | | | | CGAGTCGGTGCTTT |
| Exon 5 | Exon 5 | + | 33 | GTTTGTTCATGTACGCCGCCGTTTTAGAG |
| | SG 1 Cas9 | | | CTAGAAATAGCAAGTTAAAATAAGGCTA |
| | Scaffold | | | GTCCGTTATCAACTTGAAAAAGTGGCAC |
| | | | | CGAGTCGGTGCTTT |
| Exon 5 | Exon 5 | + | 34 | TGTTCATGTACGCCGCCTGGGTTTTAGAG |
| | SG 2 Cas9 | | | CTAGAAATAGCAAGTTAAAATAAGGCTA |
| | Scaffold | | | GTCCGTTATCAACTTGAAAAAGTGGCAC |
| | | | | CGAGTCGGTGCTTT |
| Exon 5 | Exon 5 | + | 35 | GTACGCCGCCTGGTGGCAAAGTTTTAGA |
| | SG 3 Cas9 | | | GCTAGAAATAGCAAGTTAAAATAAGGCT |
| | Scaffold | | | AGTCCGTTATCAACTTGAAAAAGTGGCA |
| | | | | CCGAGTCGGTGCTTT |
| Exon 6 | Exon 6 | + | 36 | ACTGCAGCATTAAAGGCCATGTTTTAGA |
| | SG 1 Cas9 | | | GCTAGAAATAGCAAGTTAAAATAAGGCT |
| | Scaffold | | | AGTCCGTTATCAACTTGAAAAAGTGGCA |
| | | | | CCGAGTCGGTGCTTT |
| N/A | Negative | + | 37 | TGCGAATACGCCCACGCGATGTTTTAGA |
| | controls | | | GCTAGAAATAGCAAGTTAAAATAAGGCT |
| | LacZ | | | AGTCCGTTATCAACTTGAAAAAGTGGCA |
| | Cas9 | | | CCGAGTCGGTGCTTT |
| | Scaffold | | | |

Underlined: hybridization sequence; UPPER CASE only: scaffold RNA.
“MS2 Scaffold” = Scaffold containing both Cas9 binding motif and MS2 binding motif
“Cas9 Scaffold” = Cas9 binding sequence

TABLE 5
DNA sequences that encode full guide RNAs (sgRNAs) operably linked to a U6
promoter

| Target | | | | |
|---|---|---|---|---|
| Loci of | | | | |
| Human | | Target | SEQ | |
| MSH3 | Name | Strand | ID NO | Full DNA sequence (5′→3′)* |
| Exon 3 | Exon 3 SG | + | 38 | GAGGGCCTATTTCCCATGATTCCTTCATATT |
| | 1 MS2 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | CAAAGAAATGTCTGAGGACCGTTTTAGA |
| | | | | GCTAGGCCAACATGAGGATCACCCATGT |
| | | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| Exon 4 | Exon 4 SG | − | 39 | GAGGGCCTATTTCCCATGATTCCTTCATATT |
| | 1 MS2 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | GGACCGTTTGTTAGCTGATTGTTTTAGAG |
| | | | | CTAGGCCAACATGAGGATCACCCATGTC |
| | | | | TGCAGGGCCTAGCAAGTTAAAATAAGGC |
| | | | | TAGTCCGTTATCAACTTGGCCAACATGAG |
| | | | | GATCACCCATGTCTGCAGGGCCAAGTGG |
| | | | | CACCGAGTCGGTGCTTT |
| Exon 5 | Exon 5 SG | + | 40 | GAGGGCCTATTTCCCATGATTCCTTCATATT |
| | 1 MS2 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | GTTTGTTCATGTACGCCGCCGTTTTAGAG |
| | | | | CTAGGCCAACATGAGGATCACCCATGTC |
| | | | | TGCAGGGCCTAGCAAGTTAAAATAAGGC |
| | | | | TAGTCCGTTATCAACTTGGCCAACATGAG |
| | | | | GATCACCCATGTCTGCAGGGCCAAGTGG |
| | | | | CACCGAGTCGGTGCTTT |
| Exon 5 | Exon 5 SG | + | 41 | GAGGGCCTATTTCCCATGATTCCTTCATATT |
| | 2 MS2 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | TGTTCATGTACGCCGCCTGGGTTTTAGAG |
| | | | | CTAGGCCAACATGAGGATCACCCATGTC |
| | | | | TGCAGGGCCTAGCAAGTTAAAATAAGGC |
| | | | | TAGTCCGTTATCAACTTGGCCAACATGAG |
| | | | | GATCACCCATGTCTGCAGGGCCAAGTGG |
| | | | | CACCGAGTCGGTGCTTT |
| Exon 5 | Exon 5 SG | + | 42 | GAGGGCCTATTTCCCATGATTCCTTCATATT |
| | 3 MS2 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | GTACGCCGCCTGGTGGCAAAGTTTTAGA |
| | | | | GCTAGGCCAACATGAGGATCACCCATGT |
| | | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| Exon 6 | Exon 6 SG | + | 43 | GAGGGCCTATTTCCCATGATTCCTTCATATT |
| | 1 MS2 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | ACTGCAGCATTAAAGGCCATGTTTTAGA |
| | | | | GCTAGGCCAACATGAGGATCACCCATGT |
| | | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| N/A | Negative | + | 44 | GAGGGCCTATTTCCCATGATTCCTTCATATT |
| | controls | | | TGCATATACGATACAAGGCTGTTAGAGAGAT |
| | LacZ MS2 | | | AATTAGAATTAATTTGACTGTAAACACAAAGA |
| | Scaffold | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | TGCGAATACGCCCACGCGATGTTTTAGA |
| | | | | GCTAGGCCAACATGAGGATCACCCATGT |
| | | | | CTGCAGGGCCTAGCAAGTTAAAATAAGG |
| | | | | CTAGTCCGTTATCAACTTGGCCAACATGA |
| | | | | GGATCACCCATGTCTGCAGGGCCAAGTG |
| | | | | GCACCGAGTCGGTGCTTT |
| Exon 3 | Exon 3 SG | + | 45 | GAGGGCCTATTTCCCATGATTCCTTCATATT |
| | 1 Cas9 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | CAAAGAAATGTCTGAGGACCGTTTTAGA |
| | | | | GCTAGAAATAGCAAGTTAAAATAAGGCT |
| | | | | AGTCCGTTATCAACTTGAAAAAGTGGCA |
| | | | | CCGAGTCGGTGCTTT |
| Exon 4 | Exon 4 SG | − | 46 | GAGGGCCTATTTCCCATGATTCCTTCATATT |
| | 1 Cas9 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | GGACCGTTTGTTAGCTGATTGTTTTAGAG |
| | | | | CTAGAAATAGCAAGTTAAAATAAGGCTA |
| | | | | GTCCGTTATCAACTTGAAAAAGTGGCAC |
| | | | | CGAGTCGGTGCTTT |
| Exon 5 | Exon 5 SG | + | 47 | GAGGGCCTATTTCCCATGATTCCTTCATATT |
| | 1 Cas9 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | GTTTGTTCATGTACGCCGCCGTTTTAGAG |
| | | | | CTAGAAATAGCAAGTTAAAATAAGGCTA |
| | | | | GTCCGTTATCAACTTGAAAAAGTGGCAC |
| | | | | CGAGTCGGTGCTTT |
| Exon 5 | Exon 5 SG | + | 48 | GAGGGCCTATTTCCCATGATTCCTTCATATT |
| | 2 Cas9 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | TGTTCATGTACGCCGCCTGGGTTTTAGAG |
| | | | | CTAGAAATAGCAAGTTAAAATAAGGCTA |
| | | | | GTCCGTTATCAACTTGAAAAAGTGGCAC |
| | | | | CGAGTCGGTGCTTT |
| Exon 5 | Exon 5 SG | + | 49 | GAGGGCCTATTTCCCATGATTCCTTCATATT |
| | 3 Cas9 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | GTACGCCGCCTGGTGGCAAAGTTTTAGA |
| | | | | GCTAGAAATAGCAAGTTAAAATAAGGCT |
| | | | | AGTCCGTTATCAACTTGAAAAAGTGGCA |
| | | | | CCGAGTCGGTGCTTT |
| Exon 6 | Exon 6 SG | + | 50 | GAGGGCCTATTTCCCATGATTCCTTCATATT |
| | 1 Cas9 | | | TGCATATACGATACAAGGCTGTTAGAGAGAT |
| | Scaffold | | | AATTAGAATTAATTTGACTGTAAACACAAAGA |
| | | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | ACTGCAGCATTAAAGGCCAGTTTTAGAG |
| | | | | CTAGAAATAGCAAGTTAAAATAAGGCTA |
| | | | | GTCCGTTATCAACTTGAAAAAGTGGCAC |
| | | | | CGAGTCGGTGCTTT |
| Negative | Negative | + | 51 | GAGGGCCTATTTCCCATGATTCCTTCATATT |
| controls | controls | | | TGCATATACGATACAAGGCTGTTAGAGAGAT |
| | LacZ Cas9 | | | AATTAGAATTAATTTGACTGTAAACACAAAGA |
| | Scaffold | | | TATTAGTACAAAATACGTGACGTAGAAAGTA |
| | | | | ATAATTTCTTGGGTAGTTTGCAGTTTTAAAAT |
| | | | | TATGTTTTAAAATGGACTATCATATGCTTACC |
| | | | | GTAACTTGAAAGTATTTCGATTTCTTGGCTTT |
| | | | | ATATATCTTGTGGAAAGGACGAAACACCGG |
| | | | | TGCGAATACGCCCACGCGATGTTTTAGA |
| | | | | GCTAGAAATAGCAAGTTAAAATAAGGCT |
| | | | | AGTCCGTTATCAACTTGAAAAAGTGGCA |
| | | | | CCGAGTCGGTGCTTT |

*Italicized: U6 promoter; bold: added G to enhance transcription from U6 promoter; underlined: hybridization sequence; UPPER CASE only: scaffold RNA.
“MS2 Scaffold” = Scaffold containing both Cas9 binding motif and MS2 binding motif
“Cas9 Scaffold” = Cas9 binding sequence

TABLE 6
Exemplary nucleotide sequence encoding a fusion protein of a T4 DNA polymerase.

| | SEQ ID | |
|---|---|---|
| Name | NO | DNA Sequence (5′→3′) * |
| NLS-MS2-T4 mut | 77 | ATGgcttcaaactttactcagttcgtgctcgtggacaatggtgggacaggggat |
| mRNA ORF | | gtgacagtggctccttctaatttcgctaatggggggcagagtggatcagctccaa |
| | | ctcacggagccaggcctacaaggtgacatgcagcgtcaggcagtctagtgccc |
| | | agaagagaaagtataccatcaaggtggaggtccccaaagtggctacccagac |
| | | agtgggcggagtcgaactgcctgtcgccgcttggaggtcctacctgaacatggag |
| | | ctcactatcccaattttcgctaccaattctgactgtgaactcatcgtgaaggcaatgca |
| | | ggggctcctcaaagacggtaatcctatcccttccgccatcgccgctaactcaggtat |
| | | ctacagcgctggaggaggtggaagcggaggaggaggaagcggaggaggaggt |
| | | agcggacctaagaaaaagaggaaggtgAAGGAATTCTACATCA |
| | | GCATCGAGACCGTGGGTAACAACATCGTGGAAAG |
| | | ATATATTGACGAAAACGGCAAGGAGAGAACCAGA |
| | | GAGGTGGAATACCTGCCTACAATGTTCCGGCACTG |
| | | TAAAGAGGAATCCAAGTACAAGGATATCTACGGC |
| | | AAAAACTGCGCCCCTCAGAAATTCCCCAGCATGAA |
| | | AGACGCCAGAGATTGGATGAAGAGAATGGAGGAT |
| | | ATCGGACTGGAAGCCCTGGGCATGAACGATTTCAA |
| | | GCTGGCCTACATCTCCGATACATACGAAGCGAGAT |
| | | CGTGTATGATAGAAAATTCGTGCGGGTGGCCAATT |
| | | GTGACATTGAGGTGACCGGCGACAAGTTCCCTGAT |
| | | CCCATGAAAGCTGAATATGAGATCGACGCCATTAC |
| | | CCACTACGACAGCATCGACGACAGATTCTACGTGT |
| | | TCGACCTGCTGAACTCCATGTACGGCAGCGTGTCC |
| | | AAGTGGGACGCTAAGCTGGCCGCCAAGCTGGACTG |
| | | CGAGGGCGGCGACGAGGTTCCACAAGAGATCCTG |
| | | GACCGGGTCATCTACATGCCCTTCGACAACGAGAG |
| | | GGACATGCTGATGGAATACATCAACCTGTGGGAGC |
| | | AGAAGCGCCCCGCCATTTTTACAGGCTGGAACATC |
| | | GAGGGCTTCGCCGTGCCTTATATCATGAATAGAGT |
| | | GAAAATGATCCTGGGAGAACGGAGCATGAAAAGA |
| | | TTCAGCCCTATCGGCAGAGTGAAGAGCAAGCTGAT |
| | | CCAAAACATGTACGGCTCCAAGGAAATCTATAGCA |
| | | TCGATGGCGTGTCCATCCTGGATTACCTGGACCTGT |
| | | ACAAAAAGTTCGCCTTCACCAACCTGCCATCTTTCT |
| | | CTCTTGAGAGCGTCGCCCAGCACGAGACAAAGAA |
| | | GGGCAAGCTGCCGTACGACGGTCCTATCAACAAGC |
| | | TGAGAGAAACAAATCACCAGAGATACATCAGCTA |
| | | CAACATCATCGATGTGGAAAGCGTTCAGGCCATCG |
| | | ATAAAATCAGAGGCTTCATCGACCTGGTGCTGTCT |
| | | ATGTCTTACTACGCCAAGATGCCTTTTAGCGGAGT |
| | | GATGAGCCCTATCAAGACCTGGGATGCCATCATCT |
| | | TCAACAGCCTGAAGGGCGAACACAAGGTGATCCCC |
| | | CAACAGGGCAGCCACGTGAAGCAGAGCTTCCCAG |
| | | GCGCTTTTGTGTTCGAGCCCAAGCCCATAGCGCGG |
| | | AGATACATCATGAGCTTTGATCTGACCAGCCTGTA |
| | | CCCCAGCATCATTCGGCAAGTGAACATTTCTCCAG |
| | | AAACCATCAGAGGCCAGTTTAAGGTGCACCCTATC |
| | | CACGAGTATATTGCAGGCACCGCTCCTAAACCTAG |
| | | CGACGAGTACAGCTGCTCTCCTAACGGCTGGATGT |
| | | ACGACAAGCACCAGGAGGGAATCATCCCTAAGGA |
| | | AATTGCCAAGGTGTTTTTCCAGCGGAAGGACTGGA |
| | | AGAAAAAAATGTTCGCCGAGGAAATGAACGCCGA |
| | | GGCCATCAAGAAGATCATCATGAAGGGCGCCGGC |
| | | AGCTGCTCCACCAAGCCTGAGGTGGAAAGATACGT |
| | | GAAGTTCAGCGACGATTTCCTGAATGAGCTCAGCA |
| | | ACTACACCGAGTCTGTCCTGAACTCACTGATTGAG |
| | | GAATGCGAGAAGGCCGCCACCCTGGCTAATACCAA |
| | | CCAGCTGAACCGGAAGATTCTGATCAACAGCCTGT |
| | | ACGGAGCTCTGGGCAATATTCACTTCAGATACTAC |
| | | GATCTGCGAAACGCCACAGCTATTACAATTTTCGG |
| | | CCAGGTGGGCATCCAGTGGATCGCCAGAAAGATCA |
| | | ATGAGTACCTGAACAAGGTGTGCGGCACCAACGAC |
| | | GAGGACTTCATCGCCGCTGGCGATACTGATAGCGT |
| | | GTACGTTTGTGTGGACAAGGTCATCGAGAAGGTTG |
| | | GCCTGGACAGATTTAAGGAACAGAACGACCTCGTG |
| | | GAGTTCATGAACCAGTTCGGAAAGAAGAAGATGG |
| | | AACCCATGATCGATGTGGCTTATAGAGAGCTGTGC |
| | | GACTACATGAACAACAGAGAGCACCTGATGCACAT |
| | | GGATAGAGAAGCTATTTCTTGCCCTCCTCTGGGCTC |
| | | TAAGGGAGTGGGCGGATTTTGGAAAGCCAAAAAG |
| | | AGATACGCCCTGAATGTGTACGACATGGAAGATAA |
| | | GAGATTCGCCGAGCCTCACCTGAAAATCATGGGCA |
| | | TGGAAACACAGCAGAGCAGCACCCCTAAGGCTGT |
| | | GCAGGAGGCCCTGGAAGAGTCTATCCGGAGAATCT |
| | | TGCAGGAGGGCGAGGAAAGCGTGCAGGAGTACTA |
| | | CAAGAACTTCGAGAAAGAATACAGACAGCTGGAC |
| | | TACAAGGTGATCGCGGAGGTGAAGACCGCTAATG |
| | | ATATCGCCAAGTACGACGACAAGGGCTGGCCCGGC |
| | | TTCAAGTGCCCCTTCCACATCAGAGGCGTGCTCAC |
| | | CTACCGCAGAGCCGTTTCCGGCCTGGGCGTGGCCC |
| | | CTATCCTGGATGGAAACAAAGTCATGGTGCTGCCT |
| | | CTGAGAGAGGGCAACCCCTTTGGAGATAAATGCAT |
| | | CGCTTGGCCTAGCGGCACTGAGCTGCCCAAGGAAA |
| | | TCCGCTCCGACGTGCTGAGCTGGATCGATCACAGC |
| | | ACCCTGTTCCAAAAGTCCTTCGTGAAGCCCCTGGC |
| | | CGGCATGTGCGAGTCCGCCGGCATGGACTACGAGG |
| | | AAAAGGCCAGCCTGGATTTCCTGTTCGGCGGATCC |
| | | ggacctaagaaaaagaggaaggtgTGA |

*Italicized: MS2 N55K variant; bold: NLS; underlined: linker; UPPER CASE: T4 DNA polymerase.

TABLE 7
Exemplary T4 DNA polymerase version 2 amino acid sequence and a fusion protein
thereof.

| | SEQ ID | |
|---|---|---|
| Name | NO | Amino Acid Sequence (N-terminus→C-terminus) |
| T4 DNA polymerase | 78 | KEFYISIETVGNNIVERYIDENGKERTREVEYLPTMFR |
| version 2 | | HCKEESKYKDIYGKNCAPQKFPSMKDARDWMKRME |
| | | DIGLEALGMNDFKLAYISDTYGSEIVYDRKFVRVANC |
| | | DIEVTGDKFPDPMKAEYEIDAITHYDSIDDRFYVFDLL |
| | | NSMYGSVSKWDAKLAAKLDCEGGDEVPQEILDRVIY |
| | | MPFDNERDMLMEYINLWEQKRPAIFTGWNIEGFAVP |
| | | YIMNRVKMILGERSMKRFSPIGRVKSKLIQNMYGSKE |
| | | IYSIDGVSILDYLDLYKKFAFTNLPSFSLESVAQHETK |
| | | KGKLPYDGPINKLRETNHQRYISYNIIDVESVQAIDKI |
| | | RGFIDLVLSMSYYAKMPFSGVMSPIKTWDAIIFNSLK |
| | | GEHKVIPQQGSHVKQSFPGAFVFEPKPIARRYIMSFDL |
| | | TSLYPSIIRQVNISPETIRGQFKVHPIHEYIAGTAPKPSD |
| | | EYSCSPNGWMYDKHQEGIIPKEIAKVFFQRKDWKKK |
| | | MFAEEMNAEAIKKIIMKGAGSCSTKPEVERYVKFSD |
| | | DFLNELSNYTESVLNSLIEECEKAATLANTNQLNRKIL |
| | | INSLYGALGNIHFRYYDLRNATAITIFGQVGIQWIARK |
| | | INEYLNKVCGTNDEDFIAAGDTDSVYVCVDKVIEKV |
| | | GLDRFKEQNDLVEFMNQFGKKKMEPMIDVAYRELC |
| | | DYMNNREHLMHMDREAISCPPLGSKGVGGFWKAKK |
| | | RYALNVYDMEDKRFAEPHLKIMGMETQQSSTPKAV |
| | | QEALEESIRRILQEGEESVQEYYKNFEKEYRQLDYKV |
| | | IAEVKTANDIAKYDDKGWPGFKCPFHIRGVLTYRRA |
| | | VSGLGVAPILDGNKVMVLPLREGNPFGDKCIAWPSG |
| | | TELPKEIRSDVLSWIDHSTLFQKSFVKPLAGMCESAG |
| | | MDYEEKASLDFLFG |
| NLS-MS2-T4 mut | 79 | MASNFTQFVLVDNGGTGDVTVAPSNFANGVAEWISSNS |
| | | RSQAYKVTCSVROSSAQKRKYTIKVEVPKVATQTVGGVE |
| | | LPVAAWRSYLNMELTIPIFATNSDCELIVKAMQGLLKDG |
| | | NPIPSAIAANSGIYSAGGGGSGGGGSGGGGSGPKKKR |
| | | KVKEFYISIETVGNNIVERYIDENGKERTREVEYLPTM |
| | | FRHCKEESKYKDIYGKNCAPQKFPSMKDARDWMKR |
| | | MEDIGLEALGMNDFKLAYISDTYGSEIVYDRKFVRV |
| | | ANCDIEVTGDKFPDPMKAEYEIDAITHYDSIDDRFYV |
| | | FDLLNSMYGSVSKWDAKLAAKLDCEGGDEVPQEILD |
| | | RVIYMPFDNERDMLMEYINLWEQKRPAIFTGWNIEG |
| | | FAVPYIMNRVKMILGERSMKRFSPIGRVKSKLIQNMY |
| | | GSKEIYSIDGVSILDYLDLYKKFAFTNLPSFSLESVAQ |
| | | HETKKGKLPYDGPINKLRETNHQRYISYNIIDVESVQ |
| | | AIDKIRGFIDLVLSMSYYAKMPFSGVMSPIKTWDAIIF |
| | | NSLKGEHKVIPQQGSHVKQSFPGAFVFEPKPIARRYI |
| | | MSFDLTSLYPSIIRQVNISPETIRGQFKVHPIHEYIAGT |
| | | APKPSDEYSCSPNGWMYDKHQEGIIPKEIAKVFFQRK |
| | | DWKKKMFAEEMNAEAIKKIIMKGAGSCSTKPEVERY |
| | | VKFSDDFLNELSNYTESVLNSLIEECEKAATLANTNQ |
| | | LNRKILINSLYGALGNIHFRYYDLRNATAITIFGQVGI |
| | | QWIARKINEYLNKVCGTNDEDFIAAGDTDSVYVCVD |
| | | KVIEKVGLDRFKEQNDLVEFMNQFGKKKMEPMIDV |
| | | AYRELCDYMNNREHLMHMDREAISCPPLGSKGVGG |
| | | FWKAKKRYALNVYDMEDKRFAEPHLKIMGMETQQS |
| | | STPKAVQEALEESIRRILQEGEESVQEYYKNFEKEYR |
| | | QLDYKVIAEVKTANDIAKYDDKGWPGFKCPFHIRGV |
| | | LTYRRAVSGLGVAPILDGNKVMVLPLREGNPFGDKC |
| | | IAWPSGTELPKEIRSDVLSWIDHSTLFQKSFVKPLAG |
| | | MCESAGMDYEEKASLDFLFGGSGPKKKRKV |

*Italicized: MS2 N55K variant; bold: NLS; underlined: linker; non-formatted UPPER CASE: T4 DNA polymerase.

### EXAMPLES

### Example 1: Knocking Down MSH3 Gene Expression

### Rationale

Expansion of DNA repeats has been established as the causative mutation of over 40 diseases, including Huntington's disease (HD), myotonic dystrophy type 1 (DM1), fragile-X related disorders (FXDs), and several others. Somatic repeat expansion during the lifetime of an individual is regarded as a key driver of disease pathogenesis across multiple diseases. Recent human genetic evidence and data from cellular and animal models has suggested that several DNA mismatch repair genes (MSH2, MSH3, MLH1, PMS2, and MLH3) modify disease onset by promoting repeat expansion in a tissue-specific manner. Therefore, targeting these modifier genes, for example, knocking out MSH3 gene in affected tissue such as skeletal muscles and/or myocardiac tissue, is a viable therapeutic strategy for DM1, HD and other diseases.

### Approach

As a proof of principle, the experiments described herein showed the efficiency of gene knockout in a human cell line and induced pluripotent cells. Validation of the efficiency of MSH3 knockout can be performed in mice using the DMSXL mouse model to show that knocking out MSH3 block diseases progression.

### Method

### 1. Guide Design

Six MSH3 specific guides were designed specifically directed to utilizing a CasPlus system for gene editing. The CasPlus system favors editing that generates +1 frameshift and results in limited on site deletion.

The guide nucleic acids were designed to: 1) allow the generation of a STOP codon shortly after the PAM sequence in the +1 reading frame; and 2) form a 3D structure in the RNA scaffold comprising the MS2 loop and the Cas9 scaffold compatible with an editing efficient structure.

The nucleotide sequence for the hybridization sequences for each guide RNAs are shown in Table 1, and the nucleotide sequence of the scaffold RNA used in these experiments is SEQ ID NO: 8. The PAM sequence corresponding to hybridization sequences identical to SEQ ID NOs: 2, 4 and 6 is TGG, while the PAM sequence corresponding to the hybridization sequences identical to SEQ ID NOs: 1 and 5 is AGG.

The full guide RNA sequences used in these experiments are shown in SEQ ID NOs: 10-16 of Table 3, and the corresponding DNA sequences that encode for the guide RNA are shown in SEQ ID NOs: 32-38 of Table 4.

Nucleotide sequences that produce the guide RNAs were cloned into 3 plasmids:
1) Plasmid Delta T4: control plasmid coding for the guide RNA and MS2 protein (in combination with Cas9, this would allow classic CRISPR Cas9 editing);
2) Plasmid Cas-Plus v1: plasmid coding for guide RNA and the WT T4 polymerase fused with MS2 protein;
3) Plasmid Cas-Plus v2: plasmid coding for the guide RNA and the D219A mutant T4 polymerase fused with MS2 protein.

The plasmids encoding a T4 polymerase also allowed expression of a GFP protein as a reporter protein. The plasmids encoding a Cas9 protein also allowed expression of an mCherry protein as a reporter. The plasmids producing a guide RNA were operably linked to a U6 promoter. A “G” was added at the 5′UTR of the guide RNA sequence to enhance U6 promoter transcription. The full nucleotide sequences of the regions that produced the guide RNAs, including the U6 promoter, are shown in SEQ ID NO: 38-44 of Table 4.

### 2. Guide Validation: Editing Efficiency at the DNA Level Comparing Cas9 and Cas Plus

To validate the efficiency of the guides, transfection experiments were performed. Several transfection reagents were used at different concentrations. The protocol was optimized to get the highest transfection efficiency without compromising on cell viability. The final protocol was to plate HEK293 cells at 1.5×106 cells per plate and transfect them the same day with FuGENE® 4K (12 μl) and 1 μg of Cas9 encoding plasmid and 2 μg of T4 and guide encoding plasmid (ratios Fugene: DNA of 3:1). At 72-hour post transfection, the cells were sorted by flow cytometry and only double positive cells (GFP and mCherry positive) were isolated, pelleted, frozen and genomic DNA was extracted to perform sequence analysis and droplet digital PCR.

After 72 hours, cells were sorted by flow cytometry and edited cells were isolated for DNA extraction. PCR amplified fragments for each sample were analyzed by Sanger sequencing and the results were interpreted using Tide (//tide.nki.nl/).

### 3. Guide Validation: Editing Efficiency at the DNA Level Comparing Cas9 and Cas Plus

Once guides were validated based on the nucleic sequences obtained at the edited site, the 2 or 3 best guides were used for further analysis at the protein level. For this, cells were transfected, and sorted to enrich in the edited population. The edited cells were pelleted, and protein were extracted to run a western blot using the Santa Cruz Antibody #271079.

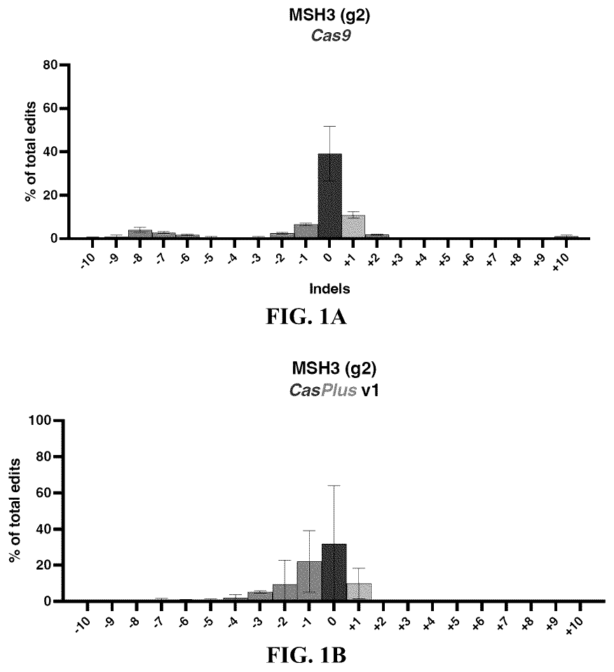

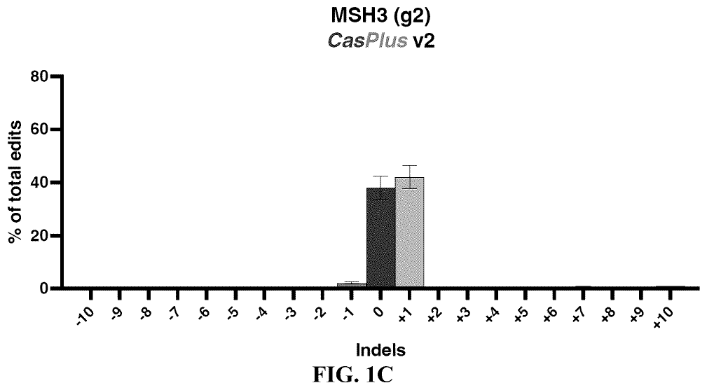

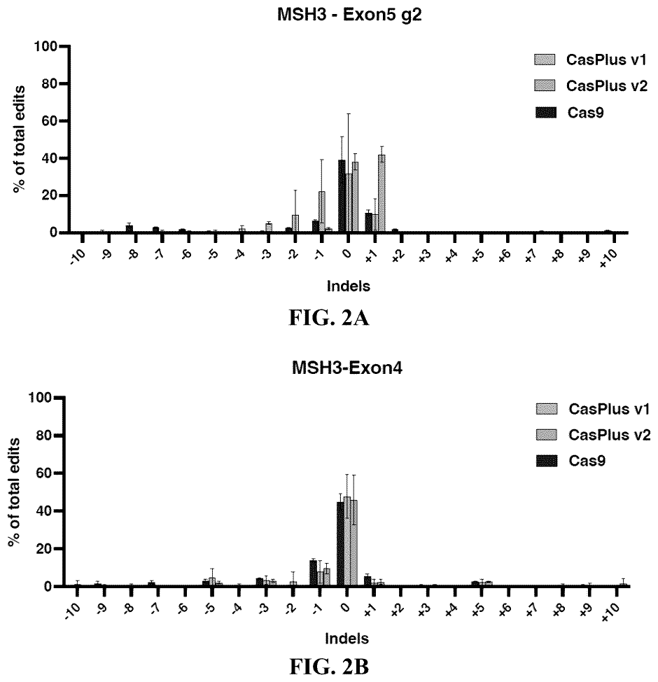

The results were shown in FIGS. 1A-1C and FIGS. 2A-2B.

As shown in FIGS. 1A-1C, the combinations of Cas9, Cas-Plusv1 or Cas-Plusv2, and the MSH3 Exon 5 sgRNA (SEQ ID NO: 14) were able to introduce indel mutations to MSH3 gene. In addition, as shown in FIG. 2A, exon 5 sgRNA+CasPlus v2 generated over 40% of +1 frameshift indels. Exon 4 sgRNA (SEQ ID NO: 12) also demonstrated editing of MSH3 gene; no difference between Cas9, CasPlus v1 or CasPlus v2 was observed when exon 4 guide was used (FIG. 2B).

4. Guide Validation: Editing Efficiency and MSH3 Knockdown by CasPlus with Electroporation.

To achieve high knockdown efficiency, transfection by electroporating Cas9 (TriLink, Catalog No. L-7206) or CasPlus mRNA composed of Cas9 mRNA in combination with T4 DNA polymerase version 2 mRNA (produced by TriLink as per T4 DNA polymerase version 2 mRNA produced by the sequence set forth in SEQ ID NO: 77, which encodes the NLS-MS2-T4 mut having the amino acid sequence set forth in SEQ ID NO: 79) was optimized. In both cases, MSH3 sgRNA having the sequence set forth in SEQ ID NO: 4 was included.

Briefly, 5 million HEK293 cells were electroporated with 1 μgCas9 mRNA, 6 μg T4 DNA polymerase v2 mRNA and 2 μM sgRNA targeting MSH3 Exon 5 guide 2 (SEQ ID NO: 14). Cells were harvested 72 hours post electroporation, followed by DNA sequencing for genomic changes, Tracking of Indels by Decomposition (TIDE) analysis, qPCR analysis for quantification of MSH3 mRNA levels, and western blot analysis to assess MSH3 protein expression.

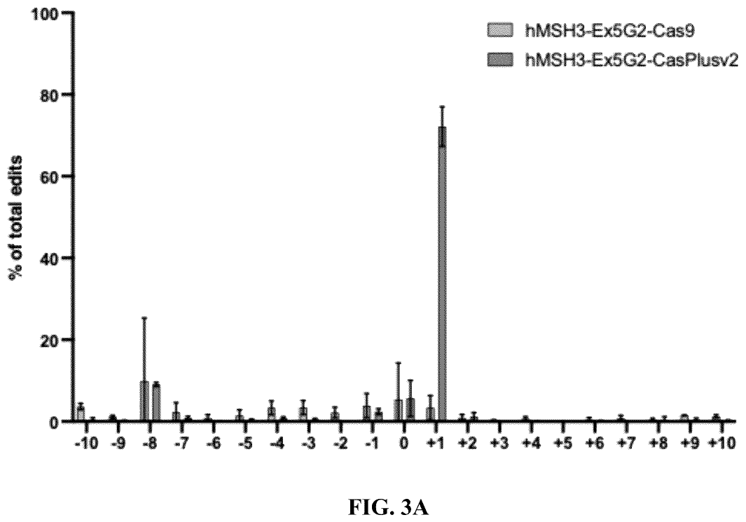

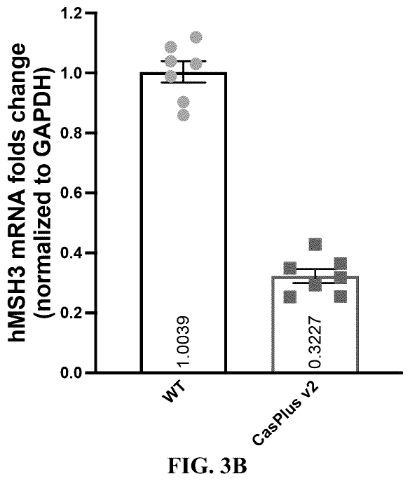

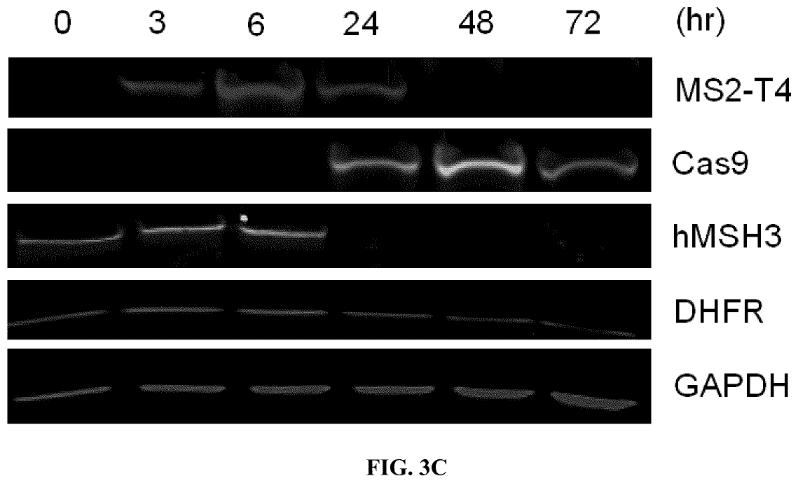

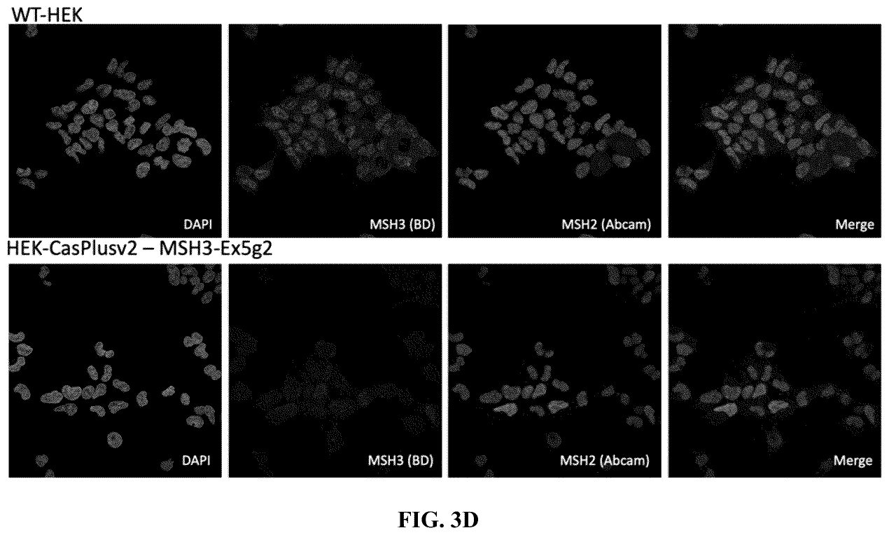

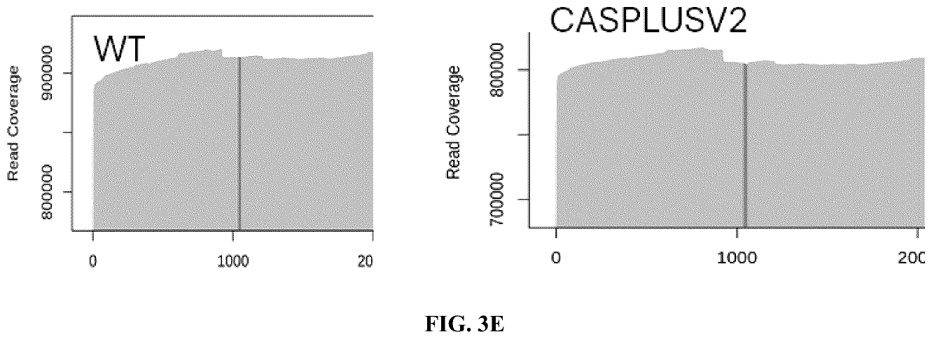

TIDE analysis showed that CasPlus editing resulted in 75.7% of cellular genomic DNAs harboring a +1 insertion and overall knockout efficiency of about 99%, whereas Cas9 edits were relatively inefficient and randomly distributed (FIG. 3A). Analysis of the MSH3 transcript levels of CasPlus edited cells also revealed that not only was the genomic DNA altered, but also that the levels of the resulting MSH3 transcript were significantly reduced, suggesting a destabilization of the mRNA as a consequence of alteration of the sequence by the +1 insertion (FIG. 3B). Consequently, MSH3 protein levels was also reduced to levels below detection limits in CasPlus edited cells, as judged by immunoblot analysis (FIG. 3C). It is noteworthy that, as per time course measurements of protein expression, MSH3 levels are reduced in lock step with the expression of the T4 DNA polymerase (which peaks at 6 hours post transfection), and Cas9 (which peaks at 48 hours post-transfection).

Loss of MSH expression was confirmed by immunofluorescence. Briefly, cells were fixed with 4% paraformaldehyde (PFA), permeabilized with 0.1% Triton-X100 and stained with anti-MSH3 antibody (BD Transduction laboratory, ref #611390) and anti MSH2 antibody (Abcam, ref #227941). Alexa fluor antibodies from Thermo Fisher were used as secondary antibodies. Nuclei were counter stained with DAPI.

Immunofluorescence microscopy of CasPlus edited cells (FIG. 3D) confirmed loss of MSH3 expression, whereas levels of its associated binding partner protein MSH2 were not affected.

Moreover, in depth genomic analysis using long range PacBio sequencing was performed, which showed the absence of large deletions at the MSH3 locus when CasPlus was used to edit the cells (FIG. 3E). Consistent with this observation, as shown in FIG. 3C, attenuation of MSH3 expression was without effect on the expression of the oppositely oriented but overlapping DHFR gene that encodes dihydrofolate reductase, an enzyme required for folate metabolism (FIG. 3C). This latter finding shows that CasPlus editing is not only site-specific, but also safe. Thus, these results validated MSH3 sgRNA exon 5 guide 2 as an efficient agent that works with CasPlus to efficiently and safely knockout MSH3 at the DNA, RNA, and protein levels.

### EQUIVALENTS AND SCOPE, INCORPORATION BY REFERENCE

Those skilled in the art will recognize, or be able to ascertain using no more than routine experimentation, many equivalents to the specific embodiments described herein. It is understood that modifications which do not substantially affect the activity of the various embodiments of this disclosure are also provided within the description of the disclosure provided herein. The scope of the present disclosure is not intended to be limited to the above description, but rather is as set forth in the appended claims.

In the claims articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members, are present in, employed in, or otherwise relevant to a given product or process.

Furthermore, it is to be understood that the disclosure encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the disclosure, or aspects of the embodiments, is/are referred to as comprising particular elements, features, steps, etc., certain embodiments of the disclosure or aspects of the embodiments consist, or consist essentially of, such elements, features, steps, etc. Thus, for each embodiment of the disclosure that comprises one or more elements, features, steps, etc., the disclosure also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the disclosure, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present disclosure can be explicitly excluded from any one or more of the claims. Where ranges are given, any value within the range can explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

Throughout this disclosure various publications, patents, and sequence database entries are mentioned. The disclosures of these publications, patents, and sequence database entries, including those items listed above, are hereby incorporated by reference in their entirety as if each individual publication or patent was specifically and individually indicated to be incorporated by reference. In case of conflict, the present application, including any definitions herein, will control.

Although the disclosure has been described with reference to the examples provided above, it should be understood that various modifications can be made without departing from the scope of the disclosure. Accordingly, the above examples are intended to illustrate but not limit the present disclosure.

1. A guide RNA comprising a first RNA sequence and a second RNA sequence, wherein the first RNA sequence is capable of hybridizing to an exon of a human MSH3 gene, and wherein the second RNA sequence comprises a Cas protein binding sequence.

2. The guide RNA of claim 1, wherein:
(a) the exon is selected from exons 3, 4, 5, and 6; and/or
(b) the first RNA sequence comprises a length of about 17 to about 23 nucleotides.

3. (canceled)

4. The guide RNA of claim 2, the first RNA sequence comprises a length of about 20 nucleotides.

5. The guide RNA of claim 1, wherein:
(a) the first RNA sequence is the reverse complement to a target sequence in the exon;
(b) the first RNA sequence comprises a nucleotide sequence having at least 90%, 95%, 99%, or 100% sequence identity to any one of SEQ ID NOs: 1-6; and/or
(c) the guide RNA comprises from 5′ to 3′ (i) the first RNA sequence and (ii) the second RNA sequence.

6. (canceled)

7. (canceled)

8. The guide RNA of claim 1, wherein the second RNA further comprises one or more MS2 bacteriophage coat protein (MS2) binding sequence.

9. The guide RNA of claim 8, wherein the second RNA sequence comprises a nucleotide sequence having at least 90%, 95%, 99%, or 100% sequence identity to SEQ ID NO: 8 or 9.

10. The guide RNA of claim 1, wherein the guide RNA comprises a nucleotide sequence having at least 90%, 95%, 99%, or 100% sequence identity to any one of SEQ ID NOs: 10-23.

11. A composition comprising:
a) the guide RNA of claim 1;
b) a Cas protein or a recombinant nucleic acid encoding the Cas protein; and
c) a T4 DNA polymerase segment or a recombinant nucleic acid encoding the T4 DNA polymerase segment.

12. The composition of claim 11, wherein:
(a) the T4 DNA polymerase segment is a fusion protein comprising an MS2 bacteriophage coat protein; and/or
(b) the fusion protein further comprises at least one nuclear localization signal sequence.

13. (canceled)

14. The composition of claim 12, wherein the T4 DNA polymerase segment and the segment of the MS2 bacteriophage coat protein are separated by a first linker sequence.

15. The composition of claim 14, further comprising a first linker amino acid sequence that links the MS2 bacteriophage coat protein to a first nuclear localization signal sequence, and a second linker sequence that links the T4 DNA polymerase segment to a second nuclear localization signal sequence.

16. The composition of claim 1, wherein:
(a) the Cas protein is Cas9 or a variant thereof; and/or
(b) the recombinant nucleic acid encoding the Cas protein and/or the recombinant nucleic acid encoding the T4 DNA polymerase segment is an mRNA.

17. (canceled)

18. A recombinant nucleic acid comprising a first nucleic acid sequence that produces the guide RNA of claim 1 when transcribed.

19. The recombinant nucleic acid of claim 18, further comprising a promoter operably linked to the first nucleic acid sequence.

20. The recombinant nucleic acid of claim 19, wherein the promoter is a U6 promoter.

21. The recombinant nucleic acid of claim 18, further comprising a nucleotide “G” between the promoter and the first nucleic acid.

22. The recombinant nucleic acid of claim 21, wherein the recombinant nucleic acid produces higher level of guide RNA than a reference recombinant nucleic acid having the same sequence as the recombinant nucleic acid but without the nucleotide “G”.

23. The recombinant nucleic acid of claim 18, further comprises a second nucleic acid sequence encoding a Cas protein and a fusion protein.

24. An expression vector comprising the recombinant nucleic acid of claim 18.

25. The expression vector of claim 24, wherein the vector is a viral vector.

26. A cell comprising the recombinant nucleic acid of any one of claim 18.

27. A pharmaceutical composition comprising the recombinant nucleic acid of claim 18, and a pharmaceutically acceptable carrier, diluent, or excipient.

28. A kit comprising the composition of any one of claim 11.

29. A system for modifying a targeted genomic locus, said system comprising the guide RNA of claim 1.

30. A method of treating a disease associated with DNA repeat expansion in a subject in need thereof, comprising introducing the composition of claim 11 to the subject, such that the disease is treated.

31. The method of claim 30, wherein:
(a) the introduction of the composition, the recombinant nucleic acid or the expression vector introduces a frameshift mutation in an exon of MSH3 in a cell of said subject; and/or
(b) the introduction of the composition, the recombinant nucleic acid or the expression vector decreases the expression of MSH3 in a cell of said subject.

32. (canceled)

33. The method of claim 31, wherein:
(a) the expression level of MSH3 is decreased by at least 10%, 20%, 30%, 40%, 50%, 60%, 70%, 80%, 90%, 95%, 99%, or 100%; and/or
(b) the decrease in expression level of MSH3 in the cell of said subject comprises a decrease in the transcript level of MSH3 in the cell of said subject.

34. (canceled)

35. The method of claim 30, wherein;
(a) the introduction of the composition, the recombinant nucleic acid or the expression vector does not change the expression of DHFR;
(b) the disease associated with DNA repeat expansion is any one of Huntington's disease (HD), myotonic dystrophy type 1 (DM1), fragile-X related disorders (FXDs), fragile XEMR (FRAXE), Friedrich's ataxia (FRDA), spinal and bulbar muscular atrophy (SBMA), spinocerebellar ataxia type 1 (SCA1), spinocerebellar ataxia type 8 (SCA8), and spinocerebellar ataxia type 12 (SCA12); and/or
(c) the DNA repeat expansion is a trinucleotide repeat expansion.

36. (canceled)

37. (canceled)

38. A pharmaceutical composition comprising the expression vector of claim 24, and a pharmaceutically acceptable carrier, diluent, or excipient.

39. A kit comprising the recombinant nucleic acid of claim 18.

40. A kit comprising the expression vector of any one of claim 24.

41. A system for modifying a targeted genomic locus, said system comprising the composition of claim 11.

42. A system for modifying a targeted genomic locus, said system comprising the recombinant nucleic acid of claim 18.

43. A system for modifying a targeted genomic locus, said system comprising the expression vector of claim 24.

44. A method of treating a disease associated with DNA repeat expansion in a subject in need thereof, comprising introducing the recombinant nucleic acid of claim 18 to the subject, such that the disease is treated.

45. A method of treating a disease associated with DNA repeat expansion in a subject in need thereof, comprising introducing the expression vector of claim 24 to the subject, such that the disease is treated.

46. A method of treating a disease associated with DNA repeat expansion in a subject in need thereof, comprising introducing the system of claim 29 to the subject, such that the disease is treated.


SEQUENCE LISTING (97 sequences)

SEQ ID NO: 1 - RNA, 20 nt, synthetic construct
  source 1..20: mol_type = other RNA
caaagaaatgtctgaggacc

SEQ ID NO: 2 - RNA, 20 nt, synthetic construct
  source 1..20: mol_type = other RNA
ggaccgtttgttagctgatt

SEQ ID NO: 3 - RNA, 20 nt, synthetic construct
  source 1..20: mol_type = other RNA
gtttgttcatgtacgccgcc

SEQ ID NO: 4 - RNA, 20 nt, synthetic construct
  source 1..20: mol_type = other RNA
tgttcatgtacgccgcctgg

SEQ ID NO: 5 - RNA, 20 nt, synthetic construct
  source 1..20: mol_type = other RNA
gtacgccgcctggtggcaaa

SEQ ID NO: 6 - RNA, 20 nt, synthetic construct
  source 1..20: mol_type = other RNA
actgcagcattaaaggccat

SEQ ID NO: 7 - RNA, 20 nt, synthetic construct
  source 1..20: mol_type = other RNA
tgcgaatacgcccacgcgat

SEQ ID NO: 8 - RNA, 139 nt, synthetic construct
  source 1..139: mol_type = other RNA
gttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 9 - RNA, 79 nt, synthetic construct
  source 1..79: mol_type = other RNA
gttttagagctagaaatagcaagttaaaataaggctagtccgttatcaacttgaaaaagtggcaccgagtcggtgcttt

SEQ ID NO: 10 - RNA, 159 nt, synthetic construct
  source 1..159: mol_type = other RNA
caaagaaatgtctgaggaccgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 11 - RNA, 159 nt, synthetic construct
  source 1..159: mol_type = other RNA
ggaccgtttgttagctgattgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 12 - RNA, 159 nt, synthetic construct
  source 1..159: mol_type = other RNA
gtttgttcatgtacgccgccgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 13 - RNA, 159 nt, synthetic construct
  source 1..159: mol_type = other RNA
tgttcatgtacgccgcctgggttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 14 - RNA, 159 nt, synthetic construct
  source 1..159: mol_type = other RNA
gtacgccgcctggtggcaaagttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 15 - RNA, 159 nt, synthetic construct
  source 1..159: mol_type = other RNA
actgcagcattaaaggccatgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 16 - RNA, 159 nt, synthetic construct
  source 1..159: mol_type = other RNA
tgcgaatacgcccacgcgatgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 17 - RNA, 99 nt, synthetic construct
  source 1..99: mol_type = other RNA
caaagaaatgtctgaggaccgttttagagctagaaatagcaagttaaaataaggctagtccgttatcaacttgaaaaagtggcaccgagtcggtgcttt

SEQ ID NO: 18 - RNA, 99 nt, synthetic construct
  source 1..99: mol_type = other RNA
ggaccgtttgttagctgattgttttagagctagaaatagcaagttaaaataaggctagtccgttatcaacttgaaaaagtggcaccgagtcggtgcttt

SEQ ID NO: 19 - RNA, 99 nt, synthetic construct
  source 1..99: mol_type = other RNA
gtttgttcatgtacgccgccgttttagagctagaaatagcaagttaaaataaggctagtccgttatcaacttgaaaaagtggcaccgagtcggtgcttt

SEQ ID NO: 20 - RNA, 99 nt, synthetic construct
  source 1..99: mol_type = other RNA
tgttcatgtacgccgcctgggttttagagctagaaatagcaagttaaaataaggctagtccgttatcaacttgaaaaagtggcaccgagtcggtgcttt

SEQ ID NO: 21 - RNA, 99 nt, synthetic construct
  source 1..99: mol_type = other RNA
gtacgccgcctggtggcaaagttttagagctagaaatagcaagttaaaataaggctagtccgttatcaacttgaaaaagtggcaccgagtcggtgcttt

SEQ ID NO: 22 - RNA, 99 nt, synthetic construct
  source 1..99: mol_type = other RNA
actgcagcattaaaggccatgttttagagctagaaatagcaagttaaaataaggctagtccgttatcaacttgaaaaagtggcaccgagtcggtgcttt

SEQ ID NO: 23 - RNA, 99 nt, synthetic construct
  source 1..99: mol_type = other RNA
tgcgaatacgcccacgcgatgttttagagctagaaatagcaagttaaaataaggctagtccgttatcaacttgaaaaagtggcaccgagtcggtgcttt

SEQ ID NO: 24 - DNA, 159 bp, synthetic construct
  source 1..159: mol_type = other DNA
caaagaaatgtctgaggaccgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 25 - DNA, 159 bp, synthetic construct
  source 1..159: mol_type = other DNA
ggaccgtttgttagctgattgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 26 - DNA, 159 bp, synthetic construct
  source 1..159: mol_type = other DNA
gtttgttcatgtacgccgccgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 27 - DNA, 159 bp, synthetic construct
  source 1..159: mol_type = other DNA
tgttcatgtacgccgcctgggttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 28 - DNA, 159 bp, synthetic construct
  source 1..159: mol_type = other DNA
gtacgccgcctggtggcaaagttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 29 - DNA, 159 bp, synthetic construct
  source 1..159: mol_type = other DNA
actgcagcattaaaggccatgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 30 - DNA, 159 bp, synthetic construct
  source 1..159: mol_type = other DNA
tgcgaatacgcccacgcgatgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 31 - DNA, 99 bp, synthetic construct
  source 1..99: mol_type = other DNA
caaagaaatgtctgaggaccgttttagagctagaaatagcaagttaaaataaggctagtccgttatcaacttgaaaaagtggcaccgagtcggtgcttt

SEQ ID NO: 32 - DNA, 99 bp, synthetic construct
  source 1..99: mol_type = other DNA
ggaccgtttgttagctgattgttttagagctagaaatagcaagttaaaataaggctagtccgttatcaacttgaaaaagtggcaccgagtcggtgcttt

SEQ ID NO: 33 - DNA, 99 bp, synthetic construct
  source 1..99: mol_type = other DNA
gtttgttcatgtacgccgccgttttagagctagaaatagcaagttaaaataaggctagtccgttatcaacttgaaaaagtggcaccgagtcggtgcttt

SEQ ID NO: 34 - DNA, 99 bp, synthetic construct
  source 1..99: mol_type = other DNA
tgttcatgtacgccgcctgggttttagagctagaaatagcaagttaaaataaggctagtccgttatcaacttgaaaaagtggcaccgagtcggtgcttt

SEQ ID NO: 35 - DNA, 99 bp, synthetic construct
  source 1..99: mol_type = other DNA
gtacgccgcctggtggcaaagttttagagctagaaatagcaagttaaaataaggctagtccgttatcaacttgaaaaagtggcaccgagtcggtgcttt

SEQ ID NO: 36 - DNA, 99 bp, synthetic construct
  source 1..99: mol_type = other DNA
actgcagcattaaaggccatgttttagagctagaaatagcaagttaaaataaggctagtccgttatcaacttgaaaaagtggcaccgagtcggtgcttt

SEQ ID NO: 37 - DNA, 99 bp, synthetic construct
  source 1..99: mol_type = other DNA
tgcgaatacgcccacgcgatgttttagagctagaaatagcaagttaaaataaggctagtccgttatcaacttgaaaaagtggcaccgagtcggtgcttt

SEQ ID NO: 38 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcaaagaaatgtctgaggaccgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 39 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggaccgtttgttagctgattgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 40 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtttgttcatgtacgccgccgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 41 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgttcatgtacgccgcctgggttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 42 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtacgccgcctggtggcaaagttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 43 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactgcagcattaaaggccatgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 44 - DNA, 410 bp, synthetic construct
  source 1..410: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgcgaatacgcccacgcgatgttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 45 - DNA, 350 bp, synthetic construct
  source 1..350: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcaaagaaatgtctgaggaccgttttagagctagaaatagcaagttaaaataaggctagtccgttatcaacttgaaaaagtggcaccgagtcggtgcttt

SEQ ID NO: 46 - DNA, 350 bp, synthetic construct
  source 1..350: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggaccgtttgttagctgattgttttagagctagaaatagcaagttaaaataaggctagtccgttatcaacttgaaaaagtggcaccgagtcggtgcttt

SEQ ID NO: 47 - DNA, 350 bp, synthetic construct
  source 1..350: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtttgttcatgtacgccgccgttttagagctagaaatagcaagttaaaataaggctagtccgttatcaacttgaaaaagtggcaccgagtcggtgcttt

SEQ ID NO: 48 - DNA, 350 bp, synthetic construct
  source 1..350: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgttcatgtacgccgcctgggttttagagctagaaatagcaagttaaaataaggctagtccgttatcaacttgaaaaagtggcaccgagtcggtgcttt

SEQ ID NO: 49 - DNA, 350 bp, synthetic construct
  source 1..350: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtacgccgcctggtggcaaagttttagagctagaaatagcaagttaaaataaggctagtccgttatcaacttgaaaaagtggcaccgagtcggtgcttt

SEQ ID NO: 50 - DNA, 349 bp, synthetic construct
  source 1..349: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggactgcagcattaaaggccagttttagagctagaaatagcaagttaaaataaggctagtccgttatcaacttgaaaaagtggcaccgagtcggtgcttt

SEQ ID NO: 51 - DNA, 350 bp, synthetic construct
  source 1..350: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgcgaatacgcccacgcgatgttttagagctagaaatagcaagttaaaataaggctagtccgttatcaacttgaaaaagtggcaccgagtcggtgcttt

SEQ ID NO: 52 - protein, 16 aa, synthetic construct
SGSETPGTSESATPES

SEQ ID NO: 53 - protein, 12 aa, synthetic construct
SGSETPGTSESA

SEQ ID NO: 54 - protein, 21 aa, synthetic construct
SGSETPGTSESATPEGGSGGS

SEQ ID NO: 55 - protein, 15 aa, synthetic construct
VPFLLEPDNINGKTC

SEQ ID NO: 56 - protein, 12 aa, synthetic construct
GSAGSAAGSGEF

SEQ ID NO: 57 - protein, 12 aa, synthetic construct
SIVAQLSRPDPA

SEQ ID NO: 58 - protein, 10 aa, synthetic construct
MKIIEQLPSA

SEQ ID NO: 59 - protein, 10 aa, synthetic construct
VRHKLKRVGS

SEQ ID NO: 60 - protein, 7 aa, synthetic construct
MSRPDPA

SEQ ID NO: 61 - protein, 4 aa, synthetic construct
GGSM

SEQ ID NO: 62 - protein, 4 aa, synthetic construct
SGGS

SEQ ID NO: 63 - protein, 6 aa, synthetic construct
PKLKRQ

SEQ ID NO: 64 - protein, 8 aa, synthetic construct
RRARRPRG

SEQ ID NO: 65 - protein, 21 aa, synthetic construct
GKRKLITSEEERSPAKRGRKS

SEQ ID NO: 66 - protein, 27 aa, synthetic construct
RSGGNHRRNGRGGRGGYNRRNNGYHPY

SEQ ID NO: 67 - protein, 27 aa, synthetic construct
TLLLRETMNNLGVSDHAVLSRKTPQPY

SEQ ID NO: 68 - protein, 21 aa, synthetic construct
GKKKKGKPGKRREQRKKKRRT

SEQ ID NO: 69 - protein, 10 aa, synthetic construct
RKHKTNRKPR

SEQ ID NO: 70 - protein, 8 aa, synthetic construct
NRRAKAKR

SEQ ID NO: 71 - protein, 6 aa, synthetic construct
RNKKKK

SEQ ID NO: 72 - protein, 5 aa, synthetic construct
RKVIK

SEQ ID NO: 73 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 74 - protein, 897 aa, synthetic construct
KEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 75 - DNA, 3414 bp, Homo sapiens
  source 1..3414: mol_type = genomic DNA
atgtctcgccggaagcctgcgtcgggcggcctcgctgcctccagctcagcccctgcgaggcaagcggttttgagccgattcttccagtctacgggaagcctgaaatccacctcctcctccacaggtgcagccgaccaggtggaccctggcgctgcagcggctgcagcggccgcagcggccgcagcgcccccagcgcccccagctcccgccttcccgccccagctgccgccgcacatagctacagaaattgacagaagaaagaagagaccattggaaaatgatgggcctgttaaaaagaaagtaaagaaagtccaacaaaaggaaggaggaagtgatctgggaatgtctggcaactctgagccaaagaaatgtctgaggaccaggaatgtttcaaagtctctggaaaaattgaaagaattctgctgcgattctgcccttcctcaaagtagagtccagacagaatctctgcaggagagatttgcagttctgccaaaatgtactgattttgatgatatcagtcttctacacgcaaagaatgcagtttcttctgaagattcgaaacgtcaaattaatcaaaaggacacaacactttttgatctcagtcagtttggatcatcaaatacaagtcatgaaaatttacagaaaactgcttccaaatcagctaacaaacggtccaaaagcatctatacgccgctagaattacaatacatagaaatgaagcagcagcacaaagatgcagttttgtgtgtggaatgtggatataagtatagattctttggggaagatgcagagattgcagcccgagagctcaatatttattgccatttagatcacaactttatgacagcaagtatacctactcacagactgtttgttcatgtacgccgcctggtggcaaaaggatataaggtgggagttgtgaagcaaactgaaactgcagcattaaaggccattggagacaacagaagttcactcttttcccggaaattgactgccctttatacaaaatctacacttattggagaagatgtgaatcccctaatcaagctggatgatgctgtaaatgttgatgagataatgactgatacttctaccagctatcttctgtgcatctctgaaaataaggaaaatgttagggacaaaaaaaagggcaacatttttattggcattgtgggagtgcagcctgccacaggcgaggttgtgtttgatagtttccaggactctgcttctcgttcagagctagaaacccggatgtcaagcctgcagccagtagagctgctgcttccttcggccttgtccgagcaaacagaggcgctcatccacagagccacatctgttagtgtgcaggatgacagaattcgagtcgaaaggatggataacatttattttgaatacagccatgctttccaggcagttacagagttttatgcaaaagatacagttgacatcaaaggttctcaaattatttctggcattgttaacttagagaagcctgtgatttgctctttggctgccatcataaaatacctcaaagaattcaacttggaaaagatgctctccaaacctgagaattttaaacagctatcaagtaaaatggaatttatgacaattaatggaacaacattaaggaatctggaaatcctacagaatcagactgatatgaaaaccaaaggaagtttgctgtgggttttagaccacactaaaacttcatttgggagacggaagttaaagaagtgggtgacccagccactccttaaattaagggaaataaatgcccggcttgatgctgtatcggaagttctccattcagaatctagtgtgtttggtcagatagaaaatcatctacgtaaattgcccgacatagagaggggactctgtagcatttatcacaaaaaatgttctacccaagagttcttcttgattgtcaaaactttatatcacctaaagtcagaatttcaagcaataatacctgctgttaattcccacattcagtcagacttgctccggaccgttattttagaaattcctgaactcctcagtccagtggagcattacttaaagatactcaatgaacaagctgccaaagttggggataaaactgaattatttaaagacctttctgacttccctttaataaaaaagaggaaggatgaaattcaaggtgttattgacgagatccgaatgcatttgcaagaaatacgaaaaatactaaaaaatccttctgcacaatatgtgacagtatcaggacaggagtttatgatagaaataaagaactctgctgtatcttgtataccaactgattgggtaaaggttggaagcacaaaagctgtgagccgctttcactctccttttattgtagaaaattacagacatctgaatcagctccgggagcagctagtccttgactgcagtgctgaatggcttgattttctagagaaattcagtgaacattatcactccttgtgtaaagcagtgcatcacctagcaactgttgactgcattttctccctggccaaggtcgctaagcaaggagattactgcagaccaactgtacaagaagaaagaaaaattgtaataaaaaatggaaggcaccctgtgattgatgtgttgctgggagaacaggatcaatatgtcccaaataatacagatttatcagaggactcagagagagtaatgataattaccggaccaaacatgggtggaaagagctcctacataaaacaagttgcattgattaccatcatggctcagattggctcctatgttcctgcagaagaagcgacaattgggattgtggatggcattttcacaaggatgggtgctgcagacaatatatataaaggacagagtacatttatggaagaactgactgacacagcagaaataatcagaaaagcaacatcacagtccttggttatcttggatgaactaggaagagggacgagcactcatgatggaattgccattgcctatgctacacttgagtatttcatcagagatgtgaaatccttaaccctgtttgtcacccattatccgccagtttgtgaactagaaaaaaattactcacaccaggtggggaattaccacatgggattcttggtcagtgaggatgaaagcaaactggatccaggcgcagcagaacaagtccctgattttgtcaccttcctttaccaaataactagaggaattgcagcaaggagttatggattaaatgtggctaaactagcagatgttcctggagaaattttgaagaaagcagctcacaagtcaaaagagctggaaggattaataaatacgaaaagaaagagactcaagtattttgcaaagttatggacgatgcataatgcacaagacctgcagaagtggacagaggagttcaacatggaagaaacacagacttctcttcttcattaa

SEQ ID NO: 76 - DNA, 564 bp, Homo sapiens
  source 1..564: mol_type = genomic DNA
atggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaa

SEQ ID NO: 77 - DNA, 3188 bp, synthetic construct
  source 1..3188: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtgaaggaattctacatcagcatcgagaccgtgggtaacaacatcgtggaaagatatattgacgaaaacggcaaggagagaaccagagaggtggaatacctgcctacaatgttccggcactgtaaagaggaatccaagtacaaggatatctacggcaaaaactgcgcccctcagaaattccccagcatgaaagacgccagagattggatgaagagaatggaggatatcggactggaagccctgggcatgaacgatttcaagctggcctacatctccgatacatacgaagcgagatcgtgtatgatagaaaattcgtgcgggtggccaattgtgacattgaggtgaccggcgacaagttccctgatcccatgaaagctgaatatgagatcgacgccattacccactacgacagcatcgacgacagattctacgtgttcgacctgctgaactccatgtacggcagcgtgtccaagtgggacgctaagctggccgccaagctggactgcgagggcggcgacgaggttccacaagagatcctggaccgggtcatctacatgcccttcgacaacgagagggacatgctgatggaatacatcaacctgtgggagcagaagcgccccgccatttttacaggctggaacatcgagggcttcgccgtgccttatatcatgaatagagtgaaaatgatcctgggagaacggagcatgaaaagattcagccctatcggcagagtgaagagcaagctgatccaaaacatgtacggctccaaggaaatctatagcatcgatggcgtgtccatcctggattacctggacctgtacaaaaagttcgccttcaccaacctgccatctttctctcttgagagcgtcgcccagcacgagacaaagaagggcaagctgccgtacgacggtcctatcaacaagctgagagaaacaaatcaccagagatacatcagctacaacatcatcgatgtggaaagcgttcaggccatcgataaaatcagaggcttcatcgacctggtgctgtctatgtcttactacgccaagatgccttttagcggagtgatgagccctatcaagacctgggatgccatcatcttcaacagcctgaagggcgaacacaaggtgatcccccaacagggcagccacgtgaagcagagcttcccaggcgcttttgtgttcgagcccaagcccatagcgcggagatacatcatgagctttgatctgaccagcctgtaccccagcatcattcggcaagtgaacatttctccagaaaccatcagaggccagtttaaggtgcaccctatccacgagtatattgcaggcaccgctcctaaacctagcgacgagtacagctgctctcctaacggctggatgtacgacaagcaccaggagggaatcatccctaaggaaattgccaaggtgtttttccagcggaaggactggaagaaaaaaatgttcgccgaggaaatgaacgccgaggccatcaagaagatcatcatgaagggcgccggcagctgctccaccaagcctgaggtggaaagatacgtgaagttcagcgacgatttcctgaatgagctcagcaactacaccgagtctgtcctgaactcactgattgaggaatgcgagaaggccgccaccctggctaataccaaccagctgaaccggaagattctgatcaacagcctgtacggagctctgggcaatattcacttcagatactacgatctgcgaaacgccacagctattacaattttcggccaggtgggcatccagtggatcgccagaaagatcaatgagtacctgaacaaggtgtgcggcaccaacgacgaggacttcatcgccgctggcgatactgatagcgtgtacgtttgtgtggacaaggtcatcgagaaggttggcctggacagatttaaggaacagaacgacctcgtggagttcatgaaccagttcggaaagaagaagatggaacccatgatcgatgtggcttatagagagctgtgcgactacatgaacaacagagagcacctgatgcacatggatagagaagctatttcttgccctcctctgggctctaagggagtgggcggattttggaaagccaaaaagagatacgccctgaatgtgtacgacatggaagataagagattcgccgagcctcacctgaaaatcatgggcatggaaacacagcagagcagcacccctaaggctgtgcaggaggccctggaagagtctatccggagaatcttgcaggagggcgaggaaagcgtgcaggagtactacaagaacttcgagaaagaatacagacagctggactacaaggtgatcgcggaggtgaagaccgctaatgatatcgccaagtacgacgacaagggctggcccggcttcaagtgccccttccacatcagaggcgtgctcacctaccgcagagccgtttccggcctgggcgtggcccctatcctggatggaaacaaagtcatggtgctgcctctgagagagggcaacccctttggagataaatgcatcgcttggcctagcggcactgagctgcccaaggaaatccgctccgacgtgctgagctggatcgatcacagcaccctgttccaaaagtccttcgtgaagcccctggccggcatgtgcgagtccgccggcatggactacgaggaaaaggccagcctggatttcctgttcggcggatccggacctaagaaaaagaggaaggtgtga

SEQ ID NO: 78 - protein, 897 aa, synthetic construct
KEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFAVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 79 - protein, 1062 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFAVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGGSGPKKKRKV

SEQ ID NO: 80 - protein, 120 aa, synthetic construct
  VARIANT 1..120: note = This sequence may encompass 1-30 SGGS repeating units
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 81 - protein, 120 aa, synthetic construct
  VARIANT 1..120: note = This sequence may encompass 1-30 GGGS repeating units
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 82 - protein, 150 aa, synthetic construct
  VARIANT 1..150: note = This sequence may encompass 1-30 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 83 - protein, 30 aa, synthetic construct
  VARIANT 1..30: note = This sequence may encompass 1-30 residues
GGGGGGGGGGGGGGGGGGGGGGGGGGGGGG

SEQ ID NO: 84 - protein, 150 aa, synthetic construct
  VARIANT 1..150: note = This sequence may encompass 1-30 EAAAK repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 85 - protein, 60 aa, synthetic construct
  VARIANT 1: note = Any amino acid
  VARIANT 3: note = Any amino acid
  VARIANT 5: note = Any amino acid
  VARIANT 7: note = Any amino acid
  VARIANT 9: note = Any amino acid
  VARIANT 11: note = Any amino acid
  VARIANT 13: note = Any amino acid
  VARIANT 15: note = Any amino acid
  VARIANT 17: note = Any amino acid
  VARIANT 19: note = Any amino acid
  VARIANT 21: note = Any amino acid
  VARIANT 23: note = Any amino acid
  VARIANT 25: note = Any amino acid
  VARIANT 27: note = Any amino acid
  VARIANT 29: note = Any amino acid
  VARIANT 31: note = Any amino acid
  VARIANT 33: note = Any amino acid
  VARIANT 35: note = Any amino acid
  VARIANT 37: note = Any amino acid
  VARIANT 39: note = Any amino acid
  VARIANT 41: note = Any amino acid
  VARIANT 43: note = Any amino acid
  VARIANT 45: note = Any amino acid
  VARIANT 47: note = Any amino acid
  VARIANT 49: note = Any amino acid
  VARIANT 51: note = Any amino acid
  VARIANT 53: note = Any amino acid
  VARIANT 55: note = Any amino acid
  VARIANT 57: note = Any amino acid
  VARIANT 59: note = Any amino acid
  VARIANT 1..60: note = This sequence may encompass 1-30 XP repeating units
XPXPXPXPXPXPXPXPXPXPXPXPXPXPXPXPXPXPXPXPXPXPXPXPXPXPXPXPXPXP

SEQ ID NO: 86 - DNA, 600 bp, unidentified
  source 1..600: mol_type = unassigned DNA
cggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcgg

SEQ ID NO: 87 - DNA, 600 bp, unidentified; identical to SEQ ID NO: 86
  source 1..600: mol_type = unassigned DNA
  misc_difference 1..600: note = This sequence may encompass 55-200 cgg repeating units

SEQ ID NO: 88 - DNA, 600 bp, unidentified
  source 1..600: mol_type = unassigned DNA
ccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccgccg

SEQ ID NO: 89 - DNA, 3900 bp, unidentified
  source 1..3900: mol_type = unassigned DNA
  misc_difference 1..3900: note = This sequence may encompass 90-1300 gaa repeating units
gaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaa

SEQ ID NO: 90 - DNA, 105 bp, unidentified
  source 1..105: mol_type = unassigned DNA
cagcagcagcagcagcagcagcagcagcagcagcagcagcagcagcagcagcagcagcagcagcagcagcagcagcagcagcagcagcagcagcagcagcagcag

SEQ ID NO: 91 - DNA, 117 bp, unidentified
  source 1..117: mol_type = unassigned DNA
  misc_difference 1..117: note = This sequence may encompass 4-39 cag repeating units
cagcagcagcagcagcagcagcagcagcagcagcagcagcagcagcagcagcagcagcagcagcagcagcagcagcagcagcagcagcagcagcagcagcagcagcagcagcagcag

SEQ ID NO: 92 - DNA, 240 bp, unidentified
  source 1..240: mol_type = unassigned DNA
  misc_difference 1..240: note = This sequence may encompass 40-80 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 93 - DNA, 117 bp, unidentified; identical to SEQ ID NO: 91
  source 1..117: mol_type = unassigned DNA

SEQ ID NO: 94 - DNA, 84 bp, unidentified
  source 1..84: mol_type = unassigned DNA
  misc_difference 1..84: note = This sequence may encompass 7-28 cag repeating units
cagcagcagcagcagcagcagcagcagcagcagcagcagcagcagcagcagcagcagcagcagcagcagcagcagcagcagcag

SEQ ID NO: 95 - DNA, 234 bp, unidentified
  source 1..234: mol_type = unassigned DNA
  misc_difference 1..234: note = This sequence may encompass 55-78 cag repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 96 - protein, 12 aa, synthetic construct
GHGTGSTGSGSS

SEQ ID NO: 97 - protein, 90 aa, synthetic construct
  VARIANT 1..90: note = This sequence may encompass 1-30 GGS repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS